SEQUENCE LISTING (3 sequences)

SEQ ID NO: 1 - DNA, 20 bp, Homo sapiens
cgtgctcagggcttcttgtc

SEQ ID NO: 2 - DNA, 21 bp, Homo sapiens
ctcgtcgcccacataggaatc

SEQ ID NO: 3 - DNA, 24 bp, Homo sapiens
  misc_feature (1)..(1): FAM fluroescent dye attached
  misc_feature (24)..(24): non-fluroescent dye BHQ1 attached
tgacccatgcccaccatcacgccc

(19) United States
(12) Patent Application Publication
(10) Pub. No.: US 2022/0081705 A1
(43) Pub. Date: Mar. 17, 2022

(54) COMPOSITIONS AND METHODS FOR URINE SAMPLE STORAGE AND DNA EXTRACTION

(71) Applicant: Hangzhou New Horizon Health Technology Co. Ltd., Binjiang District, Hangzhou, Zhejiang (CN)

(72) Inventors: Yang WU, Beijing (CN); Gang LIU, Beijing (CN); Ning LU, Hangzhou, Zhejiang (CN); Yiyou CHEN, Hangzhou, Zhejiang (CN)

(21) Appl. No.: 17/420,444

(22) PCT Filed: Jan. 3, 2020
(86) PCT No.: PCT/CN2020/070292

§ 371 Date: Jul. 2, 2021

(30) Foreign Application Priority Data
Jan. 3, 2019 (CN) PCT/CN2019/070276

(51) Int. Cl. C12Q 1/6806; C12N 15/10; G01N 33/569; G01N 33/493

(52) U.S. Cl. CPC C12Q 1/6806; C12N 15/1006; G01N 2333/025; G01N 33/493; G01N 33/569

(57) ABSTRACT

The present disclosure provides compositions and methods for storing a biological sample, such as a urine sample. DNA molecules in a biological sample mixed with a storage reagent of the present disclosure can be kept stable for a surprisingly long time. In addition, also provided are compositions and methods for extracting DNA from a biological sample, such as a urine sample. Compared to commercialized products, compositions and methods of the present disclosure are more effective for DNA extraction, suitable for DNA extraction of large urine samples and easy to realize automatic DNA extraction.

### CROSS-REFERENCE

This application claims the benefit of the PCT Application No. PCT/CN2019/070276, filed Jan. 3, 2019, which application is incorporated herein by reference in its entirety.

### FIELD OF THE INVENTION

The present disclosure relates to compositions and methods for urine sample storage and DNA extraction from a urine sample.

### BACKGROUND OF THE INVENTION

As a type of convenient and simple biological sample, urine samples have been getting more and more attention in the field of molecular diagnosis and disease monitoring and treatment. In current clinical practice, the storage of urine samples mostly relies on low temperature environment, which requires additional equipment, and also leads to higher cost. The present disclosure provides compositions and methods for storing urine samples at relatively higher temperature, such as room temperature, thereby facilitating the preservation and transportation of urine samples.

The common urine DNA extraction reagents and methods can be divided into two categories. The first involves centrifugation in order to precipitate the cells in the urine, and extracting the DNA in the cell pellet. The second involves discarding the cell precipitate after centrifugation, but extracting free DNA in the supernatant. The present disclosure provides compositions and methods for simultaneously extracting free DNA and cellular DNA in urine that lead to higher DNA extraction efficiency.

Traditional DNA extraction methods include phenol chloroform method, salt-out method, NaI method and silica solid phase carrier method, but all of them have the disadvantages of complex operation, not suitable for automatic processing or samples in lag volume. On the other hand, the composition of urine samples is complex, and DNA extracted from urine samples by common DNA extraction methods often contains inhibitors that will have an impact on the application of downstream PCR. Therefore, there remains a need for 217934327 v2 developing improved DNA extraction compositions and methods that are particularly suitable for extracting DNA from urine samples.

### SUMMARY OF THE INVENTION

The present disclosure provides compositions for storing a urine sample obtained from a subject. In some embodiments, the compositions comprise, comprise essentially of, or consist of a pH buffer, a chelating agent, and a surfactant.

In some embodiments, the pH buffer is configured to adjust a pH of the composition to within a preselected range. In some embodiments, the pH buffer comprises acetic acid and a salt of acetic acid. In some embodiments, the preselected range of pH is about 5.0 to 6.5. In some embodiments, the pH of the composition is about 6.0.

In some embodiments, the salt of acetic acid is sodium acetate. In some embodiments, the sodium acetate has a concentration of about 0.5 to 1.0 mol/L, for example, about 0.5-0.7 mol/L, or about 0.6-0.7 mol/L.

In some embodiments, the chelating agent is an aminopolycarboxylic acid. In some embodiments, chelating agent is ethylenediaminetetraacetic acid (EDTA). In some embodiments, the EDTA has a concentration of about 10 to 20 mmol/L, for example, about 15-20 mmol/L, about 16-20 mmol/L or about 16-18 mmol/L.

In some embodiments, the surfactant is an anionic surfactant. In some embodiments, the anionic surfactant is a salt of dodecyl hydrogen sulfate. In some embodiments, the salt is a sodium salt, and the anionic surfactant is sodium docecyl sulfate (SDS). In some embodiments, the SDS has a concentration of about 5% to 10% (m/v), for example, about 5%-8%, about 5%-7%, about 6%-8%, or about 6%-7%.

In some embodiments, the composition does not contain a preservative, a cell fixative, or a formaldehyde quencher.

The present disclosure also provides a processed urine sample. The urine sample can be used for DNA extraction right away, or after being stored. In some embodiments, the processed urine sample comprises a urine sample collected from a subject, a pH buffer, a chelating agent, and a surfactant. In some embodiments, the pH buffer is configured to adjust a pH of the composition to within a preselected range. In some embodiments, the pH buffer comprises acetic acid and a salt of acetic acid. In some embodiments, the salt of acetic acid is sodium acetate.

In some embodiments, the preselected range of pH is about 5.0 to 6.5. In some embodiments, the pH of the composition is about 6.0. In some embodiments, the sodium acetate in the processed urine sample has a concentration of about 0.05 to 0.1 mol/L, for example, about 0.05-0.07 mol/L, or about 0.06-0.07 mol/L. In some embodiments, the chelating agent is an aminopolycarboxylic acid. In some embodiments, the chelating agent is ethylenediaminetetraacetic acid (EDTA). In some embodiments, the EDTA has a concentration of about 1 to 2.5 mmol/L, for example, about 1.5-2 mmol/L, about 1.6-2 mmol/L or about 1.6-1.8 mmol/L. In some embodiments, the surfactant is an anionic surfactant. In some embodiments, the anionic surfactant is a salt of dodecyl hydrogen sulfate. In some embodiments, the salt is a sodium salt, and the anionic surfactant is sodium docecyl sulfate (SDS). In some embodiments, the SDS has a concentration of about 0.5% to 1.5% (m/v), for example, about 0.5%-0.8%, about 0.5%-0.7%, about 0.6%-0.8%, or about 0.6%-0.7%. In some embodiments, the processed urine sample does not contain a preservative, a cell fixative, or a formaldehyde quencher.

The present disclosure further provides methods for producing a processed urine sample for storage. In some embodiments, the methods comprise mixing a urine sample collected from a subject with a pH buffer, a chelating agent, and a surfactant, or with a composition of the present disclosure as described herein.

The present disclosure further provides methods for storing a urine sample collected from a subject. In some embodiments, the methods comprise mixing the urine sample collected from the subject with a pH buffer, a chelating agent, and a surfactant, or with a composition of the present disclosure as described herein to produce a urine sample ready for storage. In some embodiments, the pH buffer, the chelating agent, and the surfactant are provided in a mixture before they are mixed with the urine sample collected from the subject, such as a composition of the present disclosure as described herein. In some embodiments, the urine sample collected from the subject contains cells of the subject and at least one viral pathogen, and both the cells and the viral pathogen are lysed after the urine sample is ready for storage. In some embodiments, the viral pathogen is a Human papillomavirus (HPV). In some embodiments, comprising storing the urine sample ready for storage at a predetermined temperature, such as 4° C., −20° C., −80° C., or at room temperature. In some embodiments, DNA content in the urine sample is stable after a 15-day to 30-day storage time. In some embodiments, DNA content in the urine sample is stable after a 1-week to 2-week storage time.

The present disclosure further provides methods for detecting the presence or absence of one or more analytes in a urine sample collected from a subject. In some embodiments, the methods comprise using a processed urine sample as described herein. In some embodiments, the analyte is a virus or any DNA molecule derive from the virus. In some embodiments, the virus is a HPV. In some embodiments, the detection of the analyte comprises detecting DNA of the virus.

The present disclosure further provides a collection of compositions and kits for extracting DNA from a urine sample of a subject. In some embodiments, the compositions or kits comprise, comprise essentially of, or consist of a lysis solution, magnetic nanoparticles, a protease, a first washing buffer, a second washing buffer, an elution buffer, or any combination thereof.

In some embodiments, the lysis solution comprises guanidinium isothiocyanate, Triton X 100, Tris-HCl, EDTA, isopropanol, or any combination thereof. In some embodiments, the guanidinium isothiocyanate has a concentration of about 2 to 6 M. In some embodiments, the Triton X 100 has a concentration of about 1 to 5%. In some embodiments, the Tris-HCl has a concentration of about 20 to 50 mM, wherein the lysis solution has a pH of about 6.5. In some embodiments, the EDTA has a concentration of about 10 to 50 mM. In some embodiments, isopropanol is added after all other components are mixed together. In some embodiments, the isopropanol has a dosage of about 50% to 200% (v/v).

In some embodiments, the guanidinium isothiocyanate has a concentration of about 2 to 6 M, the Triton X-100 has a concentration of about 1 to 5%, the Tris-HCl has a concentration of about 20 to 50 mM, the lysis solution has a pH of about 6.5, the EDTA has a concentration of about 10 to 50 mM, or any combination thereof. In some embodiments, the lysis solution comprises guanidinium isothiocyanate, Triton X-100, Tris-HCl, and EDTA. In some embodiments, the lysis solution further comprises isopropanol. In some embodiments, the isopropanol has a dosage of about 50% to 200% (v/v) of the lysis solution.

In some embodiments, the guanidinium isothiocyanate has a concentration of about 1 to 2 M, the Triton X-100 has a concentration of about 1 to 2%, the Tris-HCl has a concentration of about 5 to 10 mM, the lysis solution has a pH of about 6-7, the EDTA has a concentration of about 3 to 5 mM, the isopropanol has a volume of about 50% to 80% of the lysis solution, or any combination thereof. In some embodiments, the guanidinium isothiocyanate has a concentration of about 1.67 M, the Triton X-100 has a concentration of about 1.33%, the Tris-HCl has a concentration of about 8.33 mM, the lysis solution has a pH of about 6.5, the EDTA has a concentration of about 3.33 mM, the isopropanol has a volume of about 66.7% of the lysis solution, or any combination thereof.

In some embodiments, the magnetic nanoparticles have an inner core layer and an outer shell layer. In some embodiments, the inner core layer is composed of core-shell type magnetic nanoparticles, wherein the outer shell layer is composed of SiO₂. In some embodiments, the magnetic nanoparticles have a diameter of about 100 to 1000 nm, and a concentration of about 50 mg/ml. In some embodiments, the magnetic nanoparticles have a volume of about 10-20 μL, for example, about 20 μL.

In some embodiments, the first washing buffer comprises guanidinium isothiocyanate, Tris-HCl, NaCl, and ethanol. In some embodiments, the guanidinium isothiocyanate has a concentration of about 50 mM. In some embodiments, the Tris-HCl has a concentration of about 20 to 50 mM. In some embodiments, the first washing buffer has a pH of about 5.0. In some embodiments, the NaCl has a concentration of about 50 to 200 mM. In some embodiments, the ethanol has concentration of about 40% to 60% (v/v).

In some embodiments, the second washing buffer comprises Tris-HCl and ethanol. In some embodiments, the Tri-HCl in the second washing buffer has a concentration of about 10 to 50 mM., and the second washing buffer has a pH of about 6.0. In some embodiments, the ethanol has concentration of about 70% to 80% (v/v).

In some embodiments, the elution buffer is a Tris-EDTA buffer having a pH of about 8.0.

In some embodiments, the protease is protease K. In some embodiments, the protease K has a concentration of about 10 to 20 mg/ml. In some embodiments, the protease K has a dosage of about 2.5 to 25 μg, for example, about 25 μg.

The present disclosure further provides methods for extracting DNA from a urine sample of a subject, comprises using a kit or a collection of compositions for DNA extraction as described herein.

The present disclosure further provides methods for extracting DNA from a urine sample of a subject. In some embodiments, the methods comprise, comprise essentially of, or consist of: (1) contacting the urine sample with magnetic nanoparticles and a protease to produce a pre-treated urine sample; (2) lysing the pre-treated urine sample obtained in step (1) in a lysis solution to produce a lysed urine sample; (3) washing the magnetic nanoparticles containing DNA from urine samples obtained in step (2) with a first washing buffer; (4) washing the magnetic nanoparticles containing DNA from urine samples obtained in step (3) with a second washing buffer; (5) washing off DNA from the magnetic nanoparticles collected in step (4) with a elution buffer to obtain the extracted DNA. In some embodiments, the lysis solution, the magnetic nanoparticles, the first washing buffer, the second washing buffer, the elution buffer, the protease are those described in the present disclosure herein.

In some embodiments, step (1) in the methods for DNA extraction comprises (a) contacting the urine sample with the magnetic nanoparticles to form a mixture; (b) centrifuging the mixture or utilizing magnetic separation device to form a precipitate and a supernatant; (c) contacting the precipitate with the protease to form a reaction system; and (d) heating the reaction system under suitable conditions for a predetermined time.

In some embodiments, steps (3), (4), and/or (5) in the methods for DNA extraction comprise using a magnetic frame or an automatic nucleic acid extraction instrument.

The present disclosure provides methods for detecting the presence or absence of an analyte in a urine sample collected from a subject. In some embodiments, the methods comprises using DNA extracted from the urine sample using a kit or a collection of composition as described herein. In some embodiments, the analyte is a virus, such as a HPV. In some embodiments, the detection of the analyte comprises detecting DNA of the virus.

The present disclosure provides methods for detecting the presence or absence of an analyte in a urine sample collected from a subject. In some embodiments, the methods comprise using a processed urine sample as described herein. In some embodiments, the methods further comprise extracting DNA from the processed urine sample. In some embodiments, the step of extracting DNA from a sample comprises (a) contacting the urine sample with magnetic nanoparticles and a protease to produce a pre-treated urine sample; (b) lysing the pre-treated urine sample obtained in step (a) in a lysis solution to produce a lysed urine sample; (c) washing the magnetic nanoparticles containing DNA from urine samples obtained in step (b) with a first washing buffer; (d) washing the magnetic nanoparticles containing DNA from urine samples obtained in step (c) with a second washing buffer; (e) washing off DNA from the magnetic nanoparticles collected in step (d) with a elution buffer to obtain the extracted DNA. In some embodiments, the lysis solution, the magnetic nanoparticles, the first washing buffer, the second washing buffer, the elution buffer, the protease are those described in the present disclosure herein.

In some embodiments, the methods for detecting the presence or absence of an analyte in a urine sample of a subject further comprises treating the subject based on the presence or absence of the analyte in the urine sample.

The present disclosure further provides methods for extracting DNA from a urine sample of a subject. In some embodiments, the methods comprise using a kit as described herein. In some embodiments, the methods comprise: (1) contacting the urine sample with magnetic nanoparticles and a protease to produce a pre-treated urine sample; (2) lysing the pre-treated urine sample obtained in step (1) in a lysis solution to produce a lysed urine sample; (3) washing the magnetic nanoparticles obtained in step (2) with a first washing buffer; (4) washing the magnetic nanoparticles obtained in step (3) with a second washing buffer; (5) collecting magnetic nanoparticles in the urine sample obtained in step (4); and (6) washing off DNA from the collected magnetic nanoparticles obtained in step (5) with an elution buffer to obtain extracted DNA.

In some embodiments, the lysis solution comprises guanidinium isothiocyanate, Triton X-100, Tris-HCl, EDTA and isopropanol. In some embodiments, the guanidinium isothiocyanate has a concentration of about 1 to 2 M. In some embodiments, the Triton X 100 has a concentration of about 1 to 2%. In some embodiments, the Tris-HCl has a concentration of about 5 to 10 mM. In some embodiments, the lysis solution has a pH of about 6-7. In some embodiments, the EDTA has a concentration of about 3 to 5 mM. In some embodiments, the isopropanol has a volume of about 50% to 80% (v/v) of the lysis solution.

In some embodiments, the magnetic nanoparticles have an inner core layer and an outer shell layer, wherein the inner core layer is composed of core-shell type magnetic nanoparticles, wherein the outer shell layer is composed of SiO₂, and the magnetic nanoparticles have a diameter of about 100 to 1000 nm, and a concentration of about 50 mg/ml.

In some embodiments, the first washing buffer comprises guanidinium isothiocyanate, Tris-HCl, NaCl, and ethanol. In some embodiments, the guanidinium isothiocyanate has a concentration of about 50 to 100 mM. In some embodiments, the Tris-HCl has a concentration of about 20 to 50 mM. In some embodiments, the first washing buffer has a pH of about 5.0. In some embodiments, the NaCl has a concentration of about 50 to 200 mM. In some embodiments, the ethanol has concentration of about 40% to 60% (v/v).

In some embodiments, the second washing buffer comprises Tris-HCl and ethanol. In some embodiments, the Tri-HCl in the second washing buffer has a concentration of about 10 to 50 mM. In some embodiments, the second washing buffer has a pH of about 6.0. In some embodiments, the ethanol has a concentration of about 70% to 80% (v/v).

In some embodiments, the elution buffer is a Tris-EDTA buffer having a pH of about 8.0. In some embodiments,

In some embodiments, the protease is protease K, wherein the protease K has a concentration of about 10 to 20 mg/ml.

In some embodiments, the step (1) of the methods for extracting DNA from a urine sample (“contacting the urine sample with magnetic nanoparticles and a protease to produce a pre-treated urine sample”) comprises: (a) contacting the urine sample with the magnetic nanoparticles to form a mixture; (b) centrifuging the mixture or utilizing magnetic separation device to form a precipitate and a supernatant; (c) contacting the precipitate with the protease to form a reaction system; and (d) heating the reaction system under suitable conditions for a predetermined time.

In some embodiments, the steps of washing and/or collecting magnetic nanoparticles in the methods for extracting DNA from a urine sample of a subject comprise using a magnetic frame or an automatic nucleic acid extraction instrument.

The present disclosure further provides methods for detecting the presence or absence of an analyte in a urine sample collected from a subject. In some embodiments, the methods comprise using DNA extracted from the urine sample using a kit as described herein. In some embodiments, the analyte is a virus. In some embodiments, the virus is an HPV. In some embodiments, the detection of the analyte comprises detecting DNA of the virus.

The present disclosure further provides methods for detecting the presence or absence of an analyte in a urine sample collected from a subject. In some embodiments, the methods comprise: (1) using a processed urine sample of any one of claims 16 to 30; and (2) extracting DNA from the processed urine sample, which comprises: (a) contacting the urine sample with magnetic nanoparticles and a protease to produce a pre-treated urine sample; (b) lysing the pre-treated urine sample obtained in step (a) in a lysis solution to produce a lysed urine sample; (c) washing the magnetic nanoparticles obtained in step (b) with a first washing buffer; (d) washing themagnetic nanoparticles obtained in step (c) with a second washing buffer; (e) collecting magnetic nanoparticles in the urine sample obtained in step (d); and (f) washing off DNA from the collected magnetic nanoparticles obtained in step (e) with an elution buffer to obtain extract DNA.

In some embodiments, the lysis solution comprises guanidinium isothiocyanate, Triton X-100, Tris-HCl, EDTA, and isopropanol. In some embodiments, the guanidinium isothiocyanate has a concentration of about 1 to 2 M. In some embodiments, the Triton X 100 has a concentration of about 1 to 2%. In some embodiments, the Tris-HCl has a concentration of about 5 to 10 mM. In some embodiments, the lysis solution has a pH of about 6-7. In some embodiments, the EDTA has a concentration of about 3 to 5 mM. In some embodiments, the isopropanol has a volume of about 50% to 80% (v/v) of the lysis solution.

### BRIEF DESCRIPTION OF THE FIGURES

FIG. 1 depicts fluorescence quantitative PCR amplification curve of (3-actin gene in urine samples with or without being processed by a storage reagent of the present disclosure.

FIG. 2 depicts change of (3-actin internal standard in urine samples processed by urine storage reagent at 4° C. during 0-4 weeks after the urine samples were mixed with the urine storage reagent.

FIG. 3 depicts change of (3-actin internal standard in urine samples processed by urine storage reagent at room temperature during 0-4 weeks after the urine samples were mixed with the urine storage reagent.

FIG. 4 depicts change of HPV gene in urine samples processed by urine storage reagent at 4° C. during 0-4 weeks after the urine samples were mixed with the urine storage reagent.

FIG. 5 depicts change of HPV gene in urine samples processed by urine storage reagent at room temperature during 0-4 weeks after the urine samples were mixed with the urine storage reagent.

FIG. 6A to FIG. 6D depict amplification curves of (3-actin gene or HPV gene in DNA extracted from urine samples using different methods/kits. FIGS. 6A and 6B compare reagents and methods of the present disclosure to Quick-DNA Urine Kit (ZYMO RESEARCH, D3061). FIGS. 6C and 6D compare reagents and methods of the present disclosure to the magnetic bead urinary genomic DNA extraction kit (Enriching biotechnology, UDE-5005), and FineMag large-volume magnetic bead—DNA extraction kit for plasma free DNA (Genefine Biotech, FM107).

### DETAILED DESCRIPTION OF THE INVENTION

Compositions and Methods for Sample Storage

The present disclosure, in some embodiments, provides compositions and methods for storing a biological sample. Non-limiting examples of biological samples include, blood, sweat, tears, urine, saliva, semen, serum, plasma, cerebrospinal fluid (CSF), feces, vaginal fluid or tissue, sputum, nasopharyngeal aspirate or swab, lacrimal fluid, mucous, or epithelial swab (buccal swab), tissues, organs, bones, teeth, or tumors, among others.

In some embodiments, the biological sample is a urine sample collected from a subject. Urine samples are widely used in molecular diagnosis, as it contains cells of the subject, pathogens that are infecting the subject, or fragments and molecules of the cells and the pathogens. However, it has been challenging to store collected urine samples in a cost-effective way while maintaining stability of potentially important molecules in samples. For example, DNA molecules derived from the cells and the pathogens may degrade quickly within hours or a couple of days after the urine sample is collected, if the sample is not sored under a relatively lower temperature. Even when a urine sample is stored in a refrigerator under 4° C., the DNA molecules in the urine sample become no longer suitable for diagnosis within a couple of weeks if the urine sample is left alone without adding anything.

Compositions and methods provided in the present application are capable of protecting DNA in a biological sample from degradation. In some embodiments, the compositions can also break the cells in the sample to release the DNA in the cells and the DNA in pathogens which may be present in the sample, thereby facilitating the subsequent DNA extraction and DNA-based diagnosis. In some embodiments, the DNA is released from a pathogen. In some embodiments, the DNA is cell-free DNA in the sample. In some embodiments, the DNA is urinary circulating tumor DNA (ctDNA).

In some embodiments, a composition of the present application can be in a concentrated status before it is mixed with and diluted by a urine sample, such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can also be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. In some embodiments, based on the dilution scale, the composition is mixed with and diluted by a urine sample, so that the final working concentration (1×) in a treated urine sample is achieved.

Compositions of the present disclosure for storing urine samples comprise a pH buffer. In some embodiments, the pH buffer is a buffer suitable for biological system. In some embodiments, the pH buffer comprises ACES N-(2-Acetamido)-aminoethanesulfonic acid, AMP (2-Amino-2-methyl-1-propanol), ADA (N-(2-Acetamido)-iminodiacetic acid), BES (N,N-Bis-(2-hydroxyethyl)-2-aminoethanesulfonic acid), bicarbonate, bicine (N,N′-Bis(2-hydroxyethyl)-glycine), Bris-Tris ([Bis-(2-hydroxyethyl)-imino]-tris-(hydroxymethylmethane)), Bis-Tris-Propane (1,3-Bis [tris(hydroxymethyl)-methylamino]propane), boric acid, cacodylate (Dimethylarsinic acid), CAPS (3-(Cyclohexylamino)-propanesulfonic acid), CAP SO 3-((Cyclohexylamino)-2-hydroxy-1-propanesulfonic acid), carbonate (sodium carbonate), CHES (Cyclohexylaminoethanesulfonic acid), salt of citric acid, DIPS 0 (3-[N-Bis(hydroxyethyl)amino]-2-hydroxypropanesulfonic acid), salt of formic acid, glycine, glycylglycine, HEPES (N-(2-Hydroxyethyl)-piperazine-N′-ethanesulfonic acid), HEPPS, EPPS (N-(2-Hydroxyethyl)-piperazine-N′-3-propanesulfonic acid), HEPPSO (N-(2-Hydroxyethyl)-piperazine-N′-2-hydroxypropanesulfonic acid), imidazole, maleic acid, MES (2-(N-Morpholino)-ethanesulfonic acid), MPOS (3-(N-Morpholino)-propanesulfonic acid), POPSO (Piperazine-N,N′-bis(2-hydroxypropanesulfonic acid)), phosphate (salt of phosphoric acid), PIPES (Piperazine-N,N′-bis(2-ethanesulfonic acid)), POPSO (Piperazine-N,N′-bis(2-hydroxypropanesulfonic acid)), TAPS (3-{[Tris(hydroxymethyl)-methyl]-amino}-propanesulfonic acid), TAPSO (3-[N-Tris(hydroxymethyl)-methylamino]-2-hydroxypropanesulfonic acid), TEA (Triethanolamine), TES (24Tris(hydroxymethyl)-methylaminol-ethanesulfonic acid), Tricine (N-[Tris(hydroxymethyl)-methyl]-glycine), Tris (Tris(hydroxymethyl)-aminomethane), and acetate (salt of acetic acid). In some embodiments, the pH buffer is an acetic acid-sodium acetate system.

In some embodiments, the pH buffer is capable of maintain pH within a predetermined range after being mixed with a urine sample. In some embodiments, the predetermined pH is about 4.5 to 6.5, such as about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, and any interval among the given range. In some embodiments, the pH buffer is an acetic acid-sodium acetate system. In some embodiments, the concentration of sodium acetate in the composition can be pre-determined based on a predetermined dilution scale, and lead to a final working concentration of about 0.05 M to about 0.1 M when the composition is mixed with a urine sample, such as about 0.05 M, 0.06 M, 0.07 M, 0.08 M, 0.09 M, 0.1 M. For example, for a 10× composition, the concentration of the sodium acetate is about 0.5 M to about 1.0 M, such as 0.5 M, 0.6 M, 0.7 M, 0.8 M, 0.9 M, or 1.0 M, which can be diluted with a urine sample in a ratio of 1:9.

Compositions of the present disclosure for storing urine samples further comprise a chelating agent. As used herein, a chelating agent refers to a substance whose molecules can form several bonds to a single metal ion. Chelating agents include but are not limited to, 1,1,1-Trifluoroacetylacetone, 1,4,7-Trimethyl-1,4,7-triazacyclononane, 2,2′-Bipyrimidine, Acetylacetone, Alizarin, Amidoxime, midoxime group, Aminoethylethanolamine, Aminomethylphosphonic acid, Aminopolycarboxylic acid, ATMP, BAPTA, Bathocuproine, BDTH2, Benzotriazole, Bidentate, Bipyridine, 2,2′-Bipyridine, Bis(dicyclohexylphosphino)ethane, 1,2-Bis(dimethylarsino)benzene, 1,2-Bis(dimethylphosphino)ethane, 1,4-Bis(diphenylphosphino)butane, 1,2-Bis(diphenylphosphino)ethane, Calixarene, Carcerand, Catechol, Cavitand, Chelating resin, Chelex 100, Citrate, Citric acid, Clathrochelate, Corrole, Cryptand, 2.2.2-Cryptand, Cyclam, Cyclen, Cyclodextrin, Deferasirox, Deferiprone, Deferoxamine, Denticity, Dexrazoxane, Diacetyl monoxime, Trans-1,2-Diaminocyclohexane, 1,2-Diaminopropane, 1,5-Diaza-3,7-diphosphacyclooctanes, 1,4-Diazacycloheptane, Dibenzoylmethane, Diethylenetriamine, Diglyme, 2,3-Dihydroxybenzoic acid, Dimercaprol, 2,3-Dimercapto-1-propanesulfonic acid, Dimercaptosuccinic acid, 1,2-Dimethylethylenediamine, 1,1-Dimethylethylenediamine, Dimethylglyoxime, DIOP, Diphenylethylenediamine, 1,5-Dithiacyclooctane, Domoic acid, DOTA (chelator), DOTA-TATE, DTPMP, EDDHA, EDDS, EDTA, EDTMP, EGTA (chemical), 1,2-Ethanedithiol, Ethylenediamine, Ethylenediaminediacetic acid, Ethylenediaminetetraacetic acid, Etidronic acid, Fluo-4, Fura-2, Gallic acid, Gluconic acid, Glutamic acid, Glyoxal-bis(mesitylimine), Glyphosate, Hexafluoroacetylacetone, Homocitric acid, Iminodiacetic acid, Indo-1, Isosaccharinic acid, Kainic acid, Ligand, Malic acid, metal acetylacetonates, Metal dithiolene complex, Metallacrown, Nitrilotriacetic acid, Oxalic acid, Oxime, Pendetide, Penicillamine, Pentetic acid, Phanephos, Phenanthroline, 0-Phenylenediamine, Phosphonate, Phthalocyanine, Phytochelatin, Picolinic acid, Polyaspartic acid, Porphine, Porphyrin, 3-Pyridylnicotinamide, 4-Pyridylnicotinamide, Pyrogallol, Salicylic acid, Sarcophagine, Sodium citrate, Sodium diethyldithiocarbamate, Sodium polyaspartate, Terpyridine, Tetramethylethylenediamine, Tetraphenylporphyrin, Thenoyltrifluoroacetone, Thioglycolic acid, TPEN, 1,4,7-Triazacyclononane, Tributyl phosphate, Tridentate, Triethylenetetramine, Triphos, Trisodium citrate, 1,4,7-Trithiacyclononane, TTFA, functional variants thereof, and any combination thereof.

In some embodiments, the chelating agent is an aminopolycarboxylic acid, such as an ethylenediaminetetraacetic acid (EDTA). As used herein, the term EDTA refers to ethylenediaminetetraacetic acid or any functional derivatives thereof. In some embodiments, the EDTA concentration in the composition can be pre-determined based on a predetermined dilution scale, and lead to a final working concentration of about 1 to about 2.5 mM when the composition is mixed with and diluted by a urine sample, such as about 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, or 2.5 mM. For example, for a 10× composition, the concentration of the EDTA is about 10 to 25 mM, such as about 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, which is then diluted with a urine sample in a ratio of 1:9.

Compositions of the present disclosure for storing urine samples further comprise a surfactant. As used herein, a surfactant refers to a compound that lower the surface tension (or interfacial tension) between two liquids, between a gas and a liquid, or between a liquid and a solid. In some embodiments, the surfactant is a cationic surfactant. In some embodiments, the surfactant is a zuitterionic surfactant. In some embodiments, the surfactant is an anionic surfactant. Non-limiting examples of anionic surfactants include molecules containing anionic functional groups at their head, such as sulfate, sulfonate, phosphate, carboxylates, etc. In some embodiments, the surfactant is ammonium lauryl sulfate, sodium lauryl sulfate (sodium dodecyl sulfate, SLS, or SDS), the related alkyl-ether sulfates sodium laureth sulfate (sodium lauryl ether sulfate or SLES), sodium myreth sulfate, docusate, perfluorooctanesulfonate (PFOS), perfluorobutanesulfonate, alkyl-aryl ether phosphates, alkyl ether phosphates, sodium stearate, Triton™ X-100, Nonoxynol-9, Polysorbate, Span®, Poloxamers, Tergitol™, Antarox®, PENTEX® 99 (Dioctyl sodium sulfosuccinate (DOSS)), PFOS, Calsoft® (Linear alkylbenzene sulfonates), Texapon® (Sodium lauryl ether sulfate), Darvan® (Lignosulfonate), or any combination thereof.

In some embodiments, the surfactant is SDS. SDS concentration in the composition can be pre-determined based on a predetermined dilution scale, and lead to a final working concentration of about 0.4 to 1.5% (m/v) when the composition is mixed with and diluted by a urine sample, such as about 0.4%, 0.5%. 0.6%, 0.7%, 0.8%, 0.9%, 1.0%, 1.1%, 1.2%, 1.3%, 1.4%, 1.5%, etc. For example, for a 10× composition, the concentration of the SDS is about 4% to 15% (m/v), such as about 4%, 5%. 6%, 7%, 8%, 9%, 10%, 11%, 12%, 13%, 14%, 15%, etc., which is then diluted with a urine sample in a ratio of 1:9.

In some embodiments, compositions of the present disclosure for storing urine sample do not contain a preservative other than EDTA, a cell fixative, or a formaldehyde quencher, thus reduce total cost, and minimize potential inhibition of downstream diagnosis.

In some embodiments, instead of premixing each component mentioned above to form a reagent comprising a mixture of each component, the components mentioned above can be mixed directly with a urine sample one by one, as long as a desired final working concentration for each component is achieved.

Thus, the present disclosure also provide a processed urine sample for storage, and/or downstream DNA extraction and diagnosis. Said processed urine sample comprises urine collected from a subject in need thereof, and a pH buffer, a chelating agent, and a surfactant, as described herein.

The processed urine sample has a longer storage term compared to unprocessed urine sample collected from the same subject. In some embodiments, the diagnosis comprises detecting the presence or absence of a DNA molecule in the urine sample. In some embodiments, DNA molecules in the processed urine sample of the present disclosure are stable enough for downstream diagnosis over a long period of time. As used herein, DNA molecules in the urine sample that has been stored for a given period of time are stable if there is no significant degradation compared to DNA molecules in urine samples just collected for the same subject, so that the DNA molecules in the urine sample are in such a good quality and good quantity enough for DNA based diagnosis, such as a PCR diagnosis. In some embodiments, DNA molecules in the urine sample that has been stored for a given period of time are about 90%, about 85%, about 90%, about 91%, about 92%, about 93%, about 94%, about 95%, about 96%, about 97%, about 98%, about 99%, or more of DNA molecules in a urine sample just collected from the same subject.

In some embodiments, when the processed urine samples are stored at about −20° C., the DNA molecules in the treated urine samples are stable after about 10 days, 20 days, 30 days, 40 days, 50 days, 60 days, 70 days, 80 days, 90 days, 100 days, 200 days, 300 days, 1 year, 2 years, 3 years, 4 years, 5 years, 6 years, 7 years, 8 years, 9 years, or more after the urine samples are processed with a composition of the present application.

In some embodiments, when the processed urine samples are stored at about −20° C., the DNA molecules in the treated urine samples are stable after about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, 65 days, 70 days, 75 days, 80 days, 85 days, 90 days, 95 days, 100 days, 110 days, 120 days, 130 days, 140 days, 150 days, 200 days, 250 days, 300 days, 1 year, 2 years, 3 years, 4 years, 5 years, or more after the urine samples are processed with a composition of the present application.

In some embodiments, when the processed urine samples are stored at about 4° C., the DNA molecules in the treated urine samples are stable after about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, or more after the urine samples are processed with a composition of the present application.

In some embodiments, when the processed urine samples are stored at room temperature, the DNA molecules in the treated urine samples are stable after about 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or more after the urine samples are treated with a composition of the present application. As used herein, the term “room temperature” refers to about 15° C. to 25° C. (±2° C.).

Accordingly, the present disclosure also provides methods for producing a processed urine sample for storage under a relatively lower temperature (e.g., about 4° C., about −20° C., or about −80° C.), or under a relatively higher temperature, such as the room temperature. In some embodiments, the methods comprise mixing a urine sample collected from a subject with a pH buffer, a chelating agent, and a surfactant as described herein. In some embodiments, the methods comprise mixing a urine sample collected from a subject with a composition of the present disclosure as described herein.

The present disclosure also provides methods for storing a urine sample collected from a subject under a relatively lower temperature (e.g., about 4° C., about −20° C., or about −80° C.), or under a relatively higher temperature, such as the room temperature. In some embodiments, the methods comprise mixing a urine sample collected from a subject with a pH buffer, a chelating agent, and a surfactant as described herein, and storing the treated urine sample for a predetermined period of time. In some embodiments, the methods comprise mixing a urine sample collected from a subject with a composition of the present disclosure as described herein for a predetermined period of time. In some embodiments, the predetermined period of time is about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, 70 days, 80 days, 90 days, 100 days, 150 days, 200 days, 250 days, 300, one year, two years, three years, or more under a relatively lower temperature (e.g., about 4° C., about −20° C., or about −80° C.), In some embodiments, the period of time is about 3 days, 4 days, 5 days, 6 days, 7 days, 8 days, 9 days, 10 days, 11 days, 12 days, 13 days, 14 days, 15 days, 16 days, 17 days, 18 days, 19 days, 20 days under room temperature.

Thus, in some embodiments, a processed urine samples of the present disclosure can be stored at room temperature for at least 2 weeks, or stored at 4° C. for at least 1 month, without any significant degradation. The processed sample can be stored for an even longer time at −20° C. or −80° C.

### Compositions and Methods for DNA Extraction

The present disclosure also provides compositions and methods for extracting DNA from a biological sample collected from a subject. In some embodiments, the biological sample is collected from a mammalian subject, such as a human. In some embodiments, the biological sample is a urine sample. Non-limiting examples of biological samples include, blood, sweat, tears, urine, saliva, semen, serum, plasma, cerebrospinal fluid (CSF), feces, vaginal fluid or tissue, sputum, nasopharyngeal aspirate or swab, lacrimal fluid, mucous, or epithelial swab (buccal swab), tissues, organs, bones, teeth, or tumors, among others

Compositions and methods of the present disclosure give a simple and cost-efficient way to extract DNA from a biological sample, such as a urine sample. Particularly, compositions and methods of the present disclosure enable simultaneously extracting DNA from exfoliated cells in the biological sample, and DNA from one or more pathogen in the sample. For example, in some embodiments, the DNA extraction compositions and methods of the present disclosure can extract DNA in a urine sample more effectively. In addition, compositions and methods of the present disclosure make it possible to conduct automated DNA extraction, thus reducing labor intensity whiling increasing the overall throughput.

The urine magnetic bead extraction method provided by the present disclosure can remove PCR inhibitors well and is easy to realize automatic DNA extraction. The magnetic bead nucleic acid extraction method of the present disclosure which produces nucleic acids of high purity, is simple to operate and easy to achieve automation. In some embodiments, the methods are more useful in dealing with urine samples in large volumes, which in turn leads to higher detection sensitivity in diagnosis based on DNA molecules in urine samples.

In some embodiments, the present disclosure provides reagents for DNA extraction from a biological sample. In some embodiments, the biological sample is a urine sample. In some embodiments, the reagents comprise magnetic particles. In some embodiments, the reagents comprise a protease. In some embodiments, the reagents further comprise a lysis solution. In some embodiments, the reagents further comprise a first washing buffer. In some embodiments, the reagents further comprise a second washing buffer. In some embodiments, the reagents comprise further comprise an elution buffer. In some embodiments, said reagents can be either provided as a kit, or be provided separately before use.

In some embodiments, the magnetic particles and the protease are used to pretreat a urine sample and get it ready for DNA extraction.

In some embodiments, the lysis solution, the first washing buffer, the second washing buffer, and the elution buffer are used to extract DNA from the pretreated urine sample.

In some embodiments, DNA extraction of the present disclosure is based on magnetic particles, such as magnetic nanoparticles (e.g., magnetic nano beads).

In some embodiments, the magnetic particles have a magnetic core, protected by a coating. The coating prevents irreversible aggregation of the magnetic particles and allows functionalization by the attachment of ligands for adsorption of DNA. In some embodiments, magnetic particles are incubated in the sample for as long as necessary to achieve optimal adsorption. In some embodiments, the magnetic particles contain iron oxide, such as Fe₃O₄ or Fe₂O₃. In some embodiments, the iron oxide material is processed into magnetic ‘pigment’ by reducing its size to few nanometers, then the magnetic ‘pigment’ can be encapsulated in non-magnetic matrices such as silica, polyvinyl alcohol (PVA), dextran, agarose, sepharose, and polystyrene, which can be biofunctionalized and used for life science applications.

In some embodiments, the magnetic particles have a core-shell structure. In some embodiments, the magnetic particles have an embedded structure.

For a core-shell structure, the magnetic particles are composed of a single superparamagnetic core with a polymer or silica surface coating, such as a magnetic core surrounded with a SiO₂ shell. In some other embodiments, the magnetic particles are composed of a polystyrene or polyvinyl alcohol (PVA) core surrounded by superparamagnetic particles and protected by a surface coating. In some embodiments, the magnetic particles have multiple layers of superparamagnetic particles alternating with encapsulation material.

For an embedded structure, superparamagnetic beads can be composed of a monodisperse matrix such as polystyrene, agarose or sepharose, which are impregnated with multiple iron-oxide nanoparticles (“magnetic pigment”). These beads are typically hundreds of nanometers in diameter and are sealed with a material that prevents loss of the magnetic pigment.

Non-limiting examples of magnetic particles for DNA extraction can be found in U.S. Pat. Nos. 6,514,688, 6,673,631, 6,027,945, 8,710,211, 6,033,878, 6,368,800, 8,324,372, 8,729,252, U.S. Application Publication Nos. 20030087286, 20150141258, 20160102305, 20130096292, 20020086326, 20050287583, 20100009351, 20110171640, 20110008797, 20180195035, 20080132694, 20040002594, 20090131650, 20160369263, 20140288398, 20030224366, and WO/2001/037291A1, WO/2001/045522A1, WO/1998/031840A1, WO/2005/021748A1, WO/2017/051939A1, WO/2017/137192A1, WO/2010/005444A1, WO/1992/008805A1, WO/2013/164319A1, WO/2015/126340A1, WO/2017/156336A1, WO/2009/102632A3, WO/2009/102632A2, WO/2009/012185A1, WO/2009/012185A9, WO/2009/115335A1, WO/2015/120445A1, WO/2015/123433A2, WO/2007/050327A2, WO/2007/050327A3, and WO/2013/028548A2, each of which is herein incorporated by reference in its entirety for all purposes.

In some embodiments, the magnetic particles are hydroxyl magnetic beads, coated by silica.

In some embodiments, the magnetic particles are magnetic beads having an average diameter of about 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or more.

Also provided is a solution containing the magnetic particles. The concentration of the magnetic particles in the solution be can predetermined as needed. In some embodiments, the concentration is about 5 mg/ml to about 100 mg/ml, about 100 mg/ml to 200 mg/ml, about 200 mg/ml to 300 mg/ml, about 300 mg/ml to 400 mg/ml, about 400 mg/ml to 500 mg/ml, or more. In some embodiments, the concentration is about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 200 mg/ml, about 300 mg/ml, about 400 mg/ml, about 500 mg/ml, or more.

In some embodiments, the solution containing the magnetic particles is mixed with a sample containing DNA. In some embodiments, the final concertation of the magnetic particles after mixed with the sample is predetermined, based on potential or actual quantity of DNA in the sample. In some embodiments, the final working concentration of the magnetic particles after being mixed with the sample containing DNA is about 0.01 to 0.5 mg/ml. In some embodiments, the final working concentration is about 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.25 mg/ml, 0.3 mg/ml, 0.35 mg/ml, 0.4 mg/ml, 0.45 mg/ml, 0.5 mg/ml, or more.

In some embodiments, after the magnetic particles is mixed with a sample containing DNA are mixed, the mixture is shaken for a predetermined time. In some embodiments, optionally the mixture is set still for a certain period of time after being mixed. The mixture is then centrifuged at a predetermined speed to precipitate the magnetic particles. In some embodiments, the supernatant is removed and the precipitated magnetic particles is processed further for DNA extraction.

In some embodiments, the precipitated magnetic particles are processed by a protease. In some embodiments, the protease is a broad-spectrum protease. In some embodiments, the protease is a serine protease, a cysteine protease, a threonine protease, an aspartic protease, a glutamic protease, a metalloprotease, an asparagine peptide lyase.

In some embodiments, the serine protease is protease K (EC 3.4.21.64, proteinase K, endopeptidase K, Tritirachium alkaline proteinase, Tritirachium album serine proteinase, Tritirachium album proteinase K). In some embodiments, the term protease K also include any functional variants of a natural protease K.

Also provided is a solution containing a protease, such as protease K. The concentration of the protease in the solution be can predetermined as needed. In some embodiments, the concentration is about 1 mg/ml to about 100 mg/ml. In some embodiments, the concentration is about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, or more.

In some embodiments, the precipitated magnetic particles are mixed with a solution comprising a protease, such as protease K. In some embodiments, the final concertation of the protease after mixed is predetermined. In some embodiments, the final working concentration of the protease after being mixed with the precipitated magnetic particle is about 5 to 500 μg/ml. In some embodiments, the final working concentration is about 5 μg/ml, 6 μg/ml, 7 μg/ml, 8 μg/ml, 9 μg/ml, 10 μg/ml, 50 μg/ml, 100 μg/ml, 150 μg/ml, 200 μg/ml, 250 μg/ml, 300 μg/ml, 350 μg/ml, 400 μg/ml, 450 μg/ml, 500n/ml, or more.

In some embodiments, the mixture of precipitated magnetic particles and the protease can be set still at a desired temperature for a predetermined time. In some embodiments, the desired temperature is the preferred enzymatic reaction temperature of the protease. In some embodiments, the protease is protease K, and the temperature is about 20° C. to about 60° C. In some embodiments, the temperature is about 50° C. to about 60° C. In some embodiments, the temperature is about 55° C. (±2° C.).

In some embodiments, the mixture of precipitated magnetic particles and the protease can be set still for a predetermined period of time. In some embodiments, the time is about 5 min, about 10 min, about 15 min, about 20 min, about 25 min, about 30 min, about 35 min, about 40 min, about 45 min, about 50 min, about 55 min, about 60 min, about 1.5 hour, about 2 hours, about 3 hours, about 4 hours, about 5 hours, or more.

In some embodiments, after the urine sample is pretreated with the magnetic particles and the protease, it is brought to the next stage for DNA extraction. In some embodiments, a lysis solution, a first washing buffer, a second washing buffer, and an elution buffer are used sequentially.

In some embodiments, the lysis solution comprises a compound having the structure of formula (I):

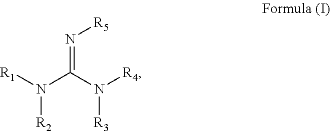

wherein R1, R2, R3, R4, and R5 are independently hydrogen, halogen, acyl, substituted acyl, alkoxycarbonyl, substituted alkoxycarbonyl, aryloxycarbonyl, substituted aryloxycarbonyl, alkyl, substituted alkyl, aryl, substituted aryl, arylalkyl, substituted arylalkyl, heteroaryl, substituted heteroaryl, heteroarylalkyl, substituted heteroarylalkyl, heteroalkyl.

In some embodiments, the compound comprises guanidinium. In some embodiments, the compound comprises guanidinium isothiocyanate, or functional derivatives thereof.

In some embodiments, the lysis solution further comprises a surfactant, a pH buffer, a chelating agent, and an alcohol (e.g., an organic compound in which the hydroxyl functional group (—OH) is bound to a carbon). In some embodiments, the surfactant is Triton X 100. In some embodiments, the pH buffer is Tris-HCl. In some embodiments, the chelating agent is EDTA. In some embodiments, the alcohol is isopropanol.

In some embodiments, the lysis solution has a pH of about 6.2 to 6.8, such as about 6.2, about 6.3, about 6.4, about 6.5, about 6.6, about 6.7, or about 6.8.

In some embodiments, a lysis solution of the present disclosure can be in a concentrated status before it is added to a sample containing DNA (e.g. a liquid sample), such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the lysis solution is mixed with a sample containing DNA, so that the final working concentration of 1× is achieved.

In some embodiments, the dilution scale is 3:1 (e.g., 3 volumes of the lysis solution is added to 1 volume of a sample containing DNA). In this case, the preparation of lysis solution comprises a) preparing a solution comprising about 2-6 M guanidinium isothiocyanate, about 1% to about 5% Triton X 100, about 20 mM to about 50 mM Tris-HCl, about 10 to about 50 mM EDTA; and b) adding to the solution about 50% to about 200% (v/v) dosage of isopropanol.

In some embodiments, after the lysis solution is mixed with a sample containing DNA, the working concentrations (1×) of each component are:

(a) about 1.0 M to 5.0M guanidinium isothiocyanate, such as about 1.0 M, about 1.5 M, about 2.0 M, about 2.5 M, about 3.0 M, about 3.5 M, about 4.0 M, about 4.5M, about 5.0 M, or more;

(b) about 0.5% to about 4% Triton X-100, such as about 0.5%, about 0.75%, about 1.0%, about 1.25%, about 1.5%, about 1.75%, about 2.0%, about 2.25%, about 2.55, about 2.75%, about 3.0%, about 3.255, about 3.5%, about 3.75%, about 4%, or more;

(c) about 5 mM to about 30 mM Tris-HCl, such as about 5 mM, about 10 mM, about 15 mM, about 20 mM, about 25 mM, about 30 mM, or more;

(d) about 2 mM to about 20 mM EDTA, such as about 2 mM, about 5 mM, about 8 mM, about 11 mM, about 14 mM, about 17 mM, about 20 mM, or more;

(e) about 30% to about 150% (v/v) dosage of isopropanol, such as about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, or more.

In some embodiments, after the sample containing the magnetic particles is mixed with the lysis solution, a container holding the mixture is shaken for a predetermined time. In some embodiments, the container is shaken for about 10 to 20 min, such as about 10 min, about 11 min, about 12 min, about 13 min, about 14 min, about 15 min, about 16 min, about 17 min, about 18 min, about 19 min, about 20 min, or more.

In some embodiments, after the sample containing the magnetic particles is lysed by the lysis solution of the present disclosure, magnetic particles in the sample are collected by using a magnetic object, such as a magnetic frame or an automatic nucleic acid extraction instrument.

In some embodiments, the collected magnetic particles are washed in a first washing buffer (washing buffer I).

In some embodiments, the first washing buffer comprises a compound having the structure of formula (I)

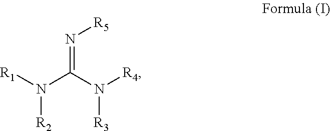

wherein R1, R2, R3, R4, and R5 are independently hydrogen, halogen, acyl, substituted acyl, alkoxycarbonyl, substituted alkoxycarbonyl, aryloxycarbonyl, substituted aryloxycarbonyl, alkyl, substituted alkyl, aryl, substituted aryl, arylalkyl, substituted arylalkyl, heteroaryl, substituted heteroaryl, heteroarylalkyl, substituted heteroarylalkyl, heteroalkyl. In some embodiments, the compound comprises guanidinium. In some embodiments, the compound comprises guanidinium isothiocyanate, or functional derivatives thereof.

In some embodiments, the first washing buffer further comprises a pH buffer, a salt, and an alcohol (e.g., an organic compound in which the hydroxyl functional group (—OH) is bound to a carbon).

In some embodiments, the pH buffer is Tris-HCl. In some embodiments, the salt is a sodium salt, such as NaCl. In some embodiments, the alcohol is ethanol.

In some embodiments, the first washing buffer has a pH of about 4.5 to 5.5, such as about 4.5, about 4.6, about 4.7, about 4.8, about 4.9, about 5.0, about 5.0, about 5.1, about 5.2, about 5.3, about 5.4, about 5.5.

In some embodiments, the first washing buffer of the present disclosure can be in a concentrated status before it is used to wash the magnetic particles, such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the washing buffer is diluted by a suitable solvent, so that the final working concentration is achieved.

The working concentrations of each component are:

(a) about 50 to about 100 mM guanidinium isothiocyanate, such as about 50 mM, about 55 mM, about 60 mM, about 65 mM, about 70 mM, about 75 mM, about 80 mM, about 85 mM, about 90 mM, about 95 mM, about 100 mM, or more;

(b) about 20 mM to about 50 mM Tris-HCl, such as about 20 mM, about 25 mM, about 30 mM, about 35 mM, about 40 mM, about 45 mM, about 50 mM, or more;

(c) about 50 mM to about 200 mM NaCl, such as about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, or more;

and

(d) about 40% to about 60% (v/v) ethanol, such as about 40%, about 45%, about 50%, about 55%, about 60%, or more.

In some embodiments, for each 0.1 mg to 1 mg magnetic particles, about 500 to 1000 μl first washing buffer is used.

In some embodiments, the magnetic particles in the sample are washed for a predetermined period of time. In some embodiments, the magnetic particles are washed for about 1 to 10 min, such as about 1 min, about 2 min, about 3 min, about 4 min, about 5 min, about 6 min, about 7 min, about 8 min, about 9 min, about 10 min, or more.

After the magnetic particles have been washed in the first washing buffer, the magnetic particles are collected again by using a magnetic object, such as a magnetic frame or an automatic nucleic acid extraction instrument.

In some embodiments, the collected magnetic particles are washed in a second washing buffer (washing buffer II).

In some embodiments, the second washing buffer further comprises a pH buffer, and an alcohol (e.g., an organic compound in which the hydroxyl functional group (—OH) is bound to a carbon).

In some embodiments, the pH buffer is Tris-HCl. In some embodiments, the alcohol is ethanol.

In some embodiments, the second washing buffer has a pH of about 5.5 to 6.5, such as about 5.5, about 5.6, about 5.7, about 5.8, about 5.9, about 6.0, about 6.1, about 6.2, about 6.3, about 6.4, about 6.5.

In some embodiments, the second washing buffer of the present disclosure can be in a concentrated status before it is used to wash the magnetic particles, such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the washing buffer is diluted by a suitable solvent, so that the final working concentration is achieved.

In some embodiments, the working concentrations of each component are:

(a) about 10 mM to about 50 mM Tris-HCl, such as about 10 mM, about 15 mM, about 20 mM, about 25 mM, about 30 mM, about 35 mM, about 40 mM, about 45 mM, about 50 mM, or more; and

(b) about 70% to 80% ethanol, such as about 71%, about 72%, about 72%, about 73%, about 74%, about 75%, about 76%, about 77%, about 78%, about 79%, about 80%.

In some embodiments, for each 0.1 mg to 1 mg magnetic particles, about 500 to 1000 μl second washing buffer is used.

In some embodiments, the magnetic particles in the sample are washed in the second washing buffer for a predetermined period of time. In some embodiments, the magnetic particles are washed for about 1 to 10 min, such as about 1 min, about 2 min, about 3 min, about 4 min, about 5 min, about 6 min, about 7 min, about 8 min, about 9 min, about 10 min, or more.

In some embodiments, after being washed with the second washing buffer, the magnetic particles are collected again by using a magnetic object, such as a magnetic frame or an automatic nucleic acid extraction instrument.

In some embodiments, the collected magnetic particles are treated in an elution buffer to release the isolated DNA molecules.

In some embodiments, the elution buffer is a TE buffer. In some embodiments, the TE buffer is a 1×TE buffer comprises about 10 mM Tris and about 1 mM EDTA. In some embodiments, the pH of the TE buffer is brought to about 8.0 with HCl.

In some embodiments, before the magnetic particles are treated by the elution buffer, they are set still for a predetermined time at a preselected temperature.

In some embodiments, the predetermined time is about 1 to 10 min, such as about 1 min, about 2 min, about 3 min, about 4 min, about 5 min, about 6 min, about 7 min, about 8 min, about 9 min, about 10 min, or more.

In some embodiments, the preselected temperature can be room temperature, a higher or a lower temperature, such as about −80° C. to about 37° C.

In some embodiments, the washing-off step comprises heating the elution buffer containing the magnetic particles at a relevantly high temperature, such as about 50° C. to about 75° C., such as about 50° C., about 55° C., about 60° C., about 65° C., about 70° C., about 75° C., or more.

Kits for Urine Sample Storage and/or DNA Extraction

Kits are also provided in the present disclosure for urine sample storage, and/or for extracting DNA from a urine sample.

In some embodiments, the kits may comprise, consists of, or consist essentially of one or more components described herein that can be used to store a biological sample, such as a urine sample. In some embodiments, the kits contains a pH buffer, a chelating agent, and/or a surfactant. In some embodiments, the pH buffer is acetic acid-sodium acetate; the chelating agent is EDTA; and the surfactant is SDS. Concentrations of components in the kits for storing a urine sample are described above. In some embodiments, one or more or all components of the kits are present in a liquid form. In some embodiments, one or more or all components of the kits are present in solid form. In some embodiments, one or more components are in concentrated status and have to be diluted before using. In some embodiments, one or more components are in working concentration and can be used directly. In some embodiments, the kits contains solvent for making a solution. In some embodiments, the kits comprise a container for collecting a urine sample. In some embodiments, the kits comprise one or more measuring containers. In some embodiments, the measuring containers are used to measure the volume of the urine sample. In some embodiments, the kits comprise a container for storing the urine sample after it is mixed with the components in the kits.

In some embodiments, the kits may comprise, consists of, or consist essentially of one or more components described herein for DNA extraction, such as a lysis solution, magnetic nanoparticles, a protease, a first washing buffer, a second washing buffer, and/or an elution buffer. In some embodiments, the lysis solution comprises guanidinium isothiocyanate, Triton X 100, Tris-HCl, EDTA, and isopropanol. In some embodiments, the magnetic nanoparticles have an inner core layer and an outer shell layer, wherein the inner core layer is composed of core-shell type magnetic nanoparticles, wherein the outer shell layer is composed of SiO₂. In some embodiments, the first washing buffer comprises guanidinium isothiocyanate, Tris-HCl, NaCl, and ethanol. In some embodiments, the second washing buffer comprises Tris-HCl and ethanol. In some embodiments, the protease is protease K. In some embodiments, the elution buffer is a 1×TE buffer, having a pH of about 8.0. In some embodiments, concentrations of components in the kits are described above. In some embodiments, one or more or all components of the kits are present in a liquid form. In some embodiments, one or more or all components of the kits are present in solid form. In some embodiments, one or more components are in concentrated status and have to be diluted before using. In some embodiments, one or more components are in working concentration and can be used directly. In some embodiments, the kits contains solvent for making a solution. In some embodiments, the kits comprise one or more containers for DNA extraction. In some embodiments, the container is suitable for an automated nucleic acid extraction system. In some embodiments, the container is a multiple-well plant, such as a 48-well plant, a 96-well plate, or a 384-well plate. In some embodiments, the kits comprise a container for storing DNA extracted from the sample.

In some embodiments, the kits may comprise, consists of, or consist essentially of one or more components described herein for both storing a biological sample (e.g., a urine sample), and for extracting DNA from said biological sample.

In addition, kits of the present disclosure may include instructional materials containing directions (e.g., protocols) for the practice of the methods described herein.

### Diagnosis of Medical Conditions

Further provided are methods for detecting the presence or absence, or levels of one or more analytes in a biological sample, such as in a urine sample collected from a subject. In some embodiments, the methods comprise extracting DNA from a sample using the compositions and methods described herein, and detecting the presence or absence of the one or more analytes in the biological sample. In some embodiments, the biological sample has been processed by a composition described herein for longer storage time. In some embodiments, the processed urine sample has been stored for a period of time before it is analyzed. In some embodiments, at least one analyte is a DNA molecule in the sample. In some embodiments, the DNA molecule is a biomarker of a medical condition.

Compositions and methods of the present disclosure are suitable for the diagnosis and/or treatment of many medical conditions. In some embodiments, the medical conditions are associated with one or more organs or tissues of the genitourinary system. In some embodiments, the medical conditions are associated with pathogen infection and/or cancer. Compositions and methods of the present disclosure provide a convenient, non-invasive, and cheap way to store urine samples and to extract DNA from the urine samples. The compositions and methods also make it technically and economically possible to extract DNA from multiple samples collected from the same subject, or samples collected from multiple subjects. In addition, compositions and methods disclosed herein are suitable for large-scale automated urine sample processing and DNA extraction. Particularly, the compositions and methods are suitable for analyzing urine samples having relatively large volume (e.g., about 0.1 to 10 ml) that are collected from the same subject over a long period of time (e.g., a couple of weeks to a couple of month) without using a low temperature storage equipment, which makes it possible to conduct the analysis repetitively at a low cost, and to monitor the medical conditions in the subject. Due to the relatively larger volume of analyzed urine sample (compared to previous methods that normally deal with a urine sample having a volume less than 1 ml), the compositions and methods of the present disclosure provide more stable and reliable diagnostic results at a low cost.

Accordingly, processed samples of the present disclosure can be used for diagnosing, monitoring, and/or treatment purposes. In some embodiments, the diagnosing, monitoring, and/or treatment are concerning one or more medical conditions in the subject. In some embodiments, the medical conditions include, but are not limited to, disorders of pain; alterations in body temperature (e.g., fever); nervous system dysfunction (e.g., syncope, myalgias, movement disorders, numbness, sensory loss, delirium, dementia, memory loss, or sleep disorders); conditions associated the eyes, ears, nose, and throat; conditions associated with circulatory and/or respiratory functions (e.g., dyspinea, pulmonary edema, cough, hemoptysis, hypertension, myocardial infarctions, hypoxia, cyanosis, cardiovascular collapse, congestive heart failure, edema, or shock); conditions associated with gastrointestinal function (e.g., dysphagia, diarrhea, constipation, GI bleeding, jaundice, ascites, indigestion, nasusea, vomiting); conditions associated with renal and urinary tract function (e.g., acidosis, alkalosis, fluid and electrolyte imbalances, azotemia, or urinary abnormalities); conditions associated with sexual function and reproduction (e.g., erectile dysfunction, menstrual disturbances, hirsutism, virilization, infertility, pregnancy associated disorders and standard measurements); conditions associated with the skin (e.g., eczema, psoriasis, acne, rosacea, cutaneous infection, immunological skin diseases, or photosensitivity); conditions associated with the blood (e.g., hematology); of genes (e.g., genetic disorders); conditions associated with drug response (e.g., adverse drug responses); and of nutrition (e.g., obesity, eating disorders, or nutritional assessment). Other medical fields with which embodiments of the invention find utility include oncology (e.g., neoplasms, malignancies, angiogenesis, paraneoplasic syndromes, or oncologic emergencies); hematology (e.g., anemia, hemoactinopathies, megalooblastic anemias, hemolytic anemias, aplastic anemia, myelodysplasia, bone marrow failure, polycythemia vera, myloproliferative diseases, acute myeloid leukemia, chronic myeloid leukemia, lymphoid malignancies, plasma cell disorders, transfusion biology, or transplants); hemostasis (e.g., disorders of coagulation and thrombosis, or disorders of the platelet and vessel wall); and infectious diseases (e.g., sepsis, septic shock, fever of unknown origin, endocardidtis, bites, burns, osteomyelitis, abscesses, food poisoning, pelvic inflammatory disease, bacterial (e.g., gram positive, gram negative, miscellaneous (nocardia, actimoyces, mixed), mycobacterial, spirochetal, rickettsia, or mycoplasma); chlamydia; viral (DNA, RNA), fungal and algal infections; protozoal and helminthic infections; endocrine diseases; nutritional diseases; and metabolic diseases.

In some embodiments, the medical condition is associated with the genitourinary system. In some embodiments, the medical condition is associated with a male or a female genitourinary system. In some embodiments, the medical condition is associated with a tissue, an organ, or a part of a male genitourinary system, such as vertebral column, rectum, seminal vesicle, ejaculatory duct, anus, epididymis, testis, scrotom, ureter, urinary bladder, vas deferens, erectile tissue, penis, urethra, penis, kidneys, etc. In some embodiments, the medical condition is associated with a tissue, an organ, or a part of a female genitourinary system, such as kidneys, ureters, bladder, urethra, uterus, fallopian tubes, ovary, and vagina.

In some embodiments, the medical conditions include, but are not limited to acute glomerulonephritis, nephrotic syndrome, chronic glomerulonephritis, nephritis, nephropathy, acute renal failure, chronic renal failure, kidney infection, pyelonephritis, hydronephrosis, calculus of kidney and ureter, lower urinary tract infection, cystitis, urethritis, urethritis, urethral stricture, hyperplasia of prostate, inflammatory diseases of prostate, hydrocele, orchitis and epididymitis, redundant prepuce and phimosis, infertility, disorders of penis, benign mammary dysplasias, inflammatory disease of ovary, fallopian tube, pelvic cellular tissue, and peritoneum, inflammatory diseases of uterus, except cervix, inflammatory disease of cervix, vagina, and vulva, endometriosis, genital prolapse, disorders of uterus, sexually transmitted diseases, etc.

In some embodiments, the medical condition is associated with one or more pathogens.

In some embodiments, the pathogen is a virus. In some embodiments, the virus includes but is not limited to, Human Immunodeficiency Virus (HIV), Hepatitis B virus (HBV), Hepatitis C virus (HCV), Human papillomavirus (HPV), Herpex simplex virus (HSV), Human cytomegalovirus (HCMV), Human Herpesvirus (HHV), Human Endogenous Retrovirus (HERV), Zika virus, Dengue virus, Chikungunya virus, Ebola virus, Human T-Cell Lymphotrophic Virus, Lymphocytic choriomeningitis virus (LMCV), Epstein-Barr Virus, Varicella-Zoster Virus, JC Virus, Parvovirus, Influenza, Rotavirus, Human Adenovirus, Rubella Virus, Human Enteroviruses, chicken pox virus, mumps virus, poliovirus, echovirus, coxsackievirus, small pox virus, Vaccinia virus, Rubella virus, and Hantavirus or any other transrenalvirus. In some embodiments, the virus is a HPV.

In some embodiments, the HPV is a high-risk HPV, such as HPV types 16, 18, 31, 33, 35, 39, 45, 51, 52, 56, 58, 59, 68, 26, 53, and 66. In some embodiments, the HPV is a low-risk HPV, such as HPV types 6, 11, 42, 43, and 44.

In some embodiments, a qPCR is used for determining the presence or absence of a given HPV subtype. In some embodiments, a positive reaction is detected by accumulation of a fluorescent signal. The cycle threshold (Ct) is defined as the number of cycles required for the fluorescent signal to cross the threshold (e.g., exceeding the background level). In some embodiments, the threshold is automatically determined by the software of the qPCR instrument or other suitable methods. In some embodiments, the threshold is set just above (e.g., about 0.01%, 0.1%, 1%, 5%, or 10% higher) the terminal fluorescent value in the negative control sample. In some embodiments, when the Ct value associated with a HPV subtype amplification in a test sample is no more than (≤) about 35, 34, 33, 32, 31, 30, or less, the sample is determined as containing the HPV subtype (positive result), otherwise the sample is determined as not containing the HPV subtype (negative result). For the reference control gene amplification, when the Ct value associated with a control gene amplification in the sample is no more than (1 about 35, 34, 33, 32, 31, 30, 29 or less, the reference control gene amplification is determined to be positive, otherwise the reference control gene amplification is determined to be negative. When the reference control gene amplification is determined to be negative, and HPV gene amplification results are also negative, the test result is invalidated.

In some embodiments, the pathogen is a bacterium. In some embodiments, a bacterium is Escherichia coli, Neisseria gonorrhoeae, Leptospirosis spp., or Mycobacterium tuberculosis.

In some embodiments, the pathogen is Chlamydia trachomatis, Mycoplasma genitalium, Tricomonas vaginalis or Ureaplasma urealyticum.

In some embodiments, the medical condition is a cancer. In some embodiments, the cancer includes, but is not limited to bladder cancer, prostate cancer, ovarian cancer, uterine cancer, cervical cancer, vaginal cancer, vulvar cancer, urological cancer, kidney cancer, testicular cancer, urothelial cancer, colorectal cancer, pancreatic cancer, and gastric cancer.

In some embodiments, a processed sample of the present disclosure, such as a processed urine sample of the present disclosure can be used for diagnosing one or more medical conditions in the subject. In some embodiments, the presence or absence, or the level of one or more biomarkers associated with one or more medical conditions in the sample are determined.

Biomarkers for baldder cancer include, but are not limited to CA9, CCL18, MMP12, TMEM45A, MMP9, SEMA3D, ERBB2, CRH, and MXRA, FIXA1, apolipoprotein A1 (APOA1), apolipoprotein A2 (APOA2), peroxiredoxin 2 (PRDX2), heparin cofactor 2 precursor (HCII), serum amyloid A-4 protein (SAA4), Cystatin B, CpG islands from a promoter region selected from the group consisting of the GDF15 promoter region, HSPA2 promoter region, and TMEFF2 promoter region, ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKN1C, CFTR, COX2, DAPK1, DRG1, DRM, EDNRB, FADD, GALC, GSTP1, HNF3B, HPP1, HTERT, ICAM1, ITGA4, LAMA3, LITAF, MAGEA1, MDR1, MGMT, MINT′, MINT2, MT1 GMT, MINT′, MINT2, MT1A, MTSS1, MYOD1, OCLN, p14ARF, p16INK4a RASSF1A, RPRM, RUNX3, SALL3, SERPINBS, SLC29A1, STAT1, TMS1, TNFRSF10A, TNFRSF10C, TNFRSF10D, TNFRSF21, WWOX, and those described in U.S. Application Publication Nos. 20140303001 and 20170350894, each of which is herein incorporated by reference in its entirety for all purposes.

Biomarkers for prostate cancer include, but are not limited to, Prostate specific antigen (PSA), Prostate cancer antigen 3 (PCA3), α-methylacyl-CoA racemase, Annexin A3, TMPRSS2: ERG, Individual inflammatory cytokines (e.g., IL-6, IL-8, TGF-β1), C-reactive protein (CRP), Toll-like receptors (TLRs), Neutrophil-to-lymphocyte ratio, PD-1/PD-L1 (B7-H1), CD276 (B7-H3), CD73, Tumor-associated macrophages (TAMs), Cytotoxic CD8 tumor-infiltrating lymphocytes (TILs), Treg tumor-infiltrating lymphocytes (TILs), and those described in U.S. Pat. Nos. 8,518,650, 8,784,795, 10048265, and U.S. Application Publication Nos. 20100292331, 20170058352, 20140094380, 20140106369, 20130217647, 20130116142, 20150160224, 20130116133, 20170016903, 20130116131, 20160024592, 20100216654, 20150329912, 20180024132, 20110236910, 20130115604, 20150299807, 20160025734, 20110311998, 20160041173, 20140038838, 20090221672, 20170362663, 20050244973, 20160097082, 20150218655, 20160299145, 20150133327, 20170176443, 20160209416, 20160355887, 20150252425, 20160258958, 20180051340, 20150329911, 20080248500, 2.0150276746, 20130331279, 20110054009, 20060088894, 20140274767, 20130184169, 20150024961, 20080254481, 20070009970, 20110045053, and 20140051082, each of which is herein incorporated by reference in its entirety for all purposes.

Biomarkers for ovarian cancer include, but are not limited to, Cyr61, ApoA1, Beta-2 microglobulin, CA125, and those described in U.S. Pat. Nos. 5,769,074, 7,666,583, 8,053,198, 8,288,110, 8,206,934, 9,816,995, U.S. Application Publication Nos. US20090068690, 20090075307, 20090081685, 20140274787, 20130267439, 20070212721, 20150080229, 20180196054, 20080286814, 20110256560, 20100197561, 20150168414, 20070054329, 20100221752, 20100086948, 20060029956, 20180074063, 20100105067, 20160047815, 20090087849, 20100055690, 20150147823, 20130096022, 20170276680, 20120046185, 20150362497, 20050214760, 20110275534, 20070172902, 20140256591, 20110217238, 20180231559, 20130022998, 20150126384, 20150322530, 20120171694, 20100227335, 20150198600, 20080254048, 20140121127, 20100227343, 20160002732, 20140221240, and 20090004687, each of which is herein incorporated by reference in its entirety for all purposes.

Biomarkers for colorectal cancer includes, but are not limited to, BMP3, TFPI1, NDRG4, Septin9, TFPI2, OPLAH, FLI1, PDGFD, SFMBT2, CHST2, VAV3, DTX1, and those described in in U.S. Pat. Nos. 9,095,549, 9,835,626, 8,426,150, 10042983, and U.S. Application Publication Nos. 20090142332, 20180282815, 20050014165, 20170205414, 20130345322, 20130323253, 20120040383, 20080020940, 20100304410, 20150072341, 20120264131, 20170176441, 20120207856, 20150212088, 20080286801, 20160160296, 20180094322, 20180164320, 20140045180, 20140113829, 20130288247, 20140212415, 20090112120, 20060088862, 20130164279, 20120238463, 20150344969, 20160108476, 20150168410, 20140256586, 20150198600, 20150197819, 20160002732, 20160153054, 20120238464, 20120237929, 20140342924, 20100203522, 20150292023, 20180080937, 20120183554, 20150133330, 20120089541, 20150176082, 20130102487, 20180112273, 20140037625, 20180306800, 20170108501, and 20170234874, each of which is herein incorporated by reference in its entirety for all purposes.

Biomarkers for kidney cancer includes, but are not limited to, sorbitol, fructose, sorbitol 6-phosphate, myristate, palmitate and stearate, aquaporin-1 (AQP1), adipophilin (ADFP), and those described in U.S. Pat. Nos. 8,426,150, 8,335,550, 8,211,653, U.S. Application Publication Nos. 20160245814, 20140343865, 20100261224, 20150198600, 20060084126, 20110237450, 20170108501, 20070054282, 20170240971, 20110151460, 20170234884, 20140213475, 20180068083, 20160305947, 20150017638, 20160215349, 20120207856, 20080139402, 20030190602, 20030199685, 20100233080, 20110020836, 20170290913, 20160166685, 20160003835, 20080261258, 20180171022, 20070026415, 20150307617, 20080124329, 20100028344, 20150301058, 20160022638, 20090299154, each of which is herein incorporated by reference in its entirety for all purposes.

Biomarkers for urothelial cancer but are not limited to, but are not limited to, SLC2A1, S100A13, GAPDH, KRT17, GPRCSA, P4HA1, HSD17B2, ubiquilin 2, EGF, IL10, sTNFR, VEGF, CK18, vWF and FAS.

### Definitions

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

As used herein, the term “about” refers to plus or minus 10% or 5% of the referenced number.

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

As used herein the term “diagnosing” refers to classifying pathology, or a symptom, determining a severity of the pathology (e.g., grade or stage), monitoring pathology progression, forecasting an outcome of pathology and/or prospects of recovery.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “optionally” is used herein to mean “is provided in some embodiments and not provided in other embodiments”. Any particular embodiment of the invention may include a plurality of “optional” features unless such features conflict.

As used herein, “dosage of isopropanol (v/v)” means the ratio of the volume of isopropanol to the volume of the solution comprising all other components in the solution during the preparation of the final solution. For example, “isopropanol has a dosage of about 50% to 200% (v/v)” means that, when preparing the final solution, the volume of added isopropanol is about 50% to 200% of the volume of the solution comprising all other components in the final solution.

As used herein, the term “Ct value” refers to cycle threshold, which is the number of cycles required for the fluorescent signal to cross the threshold (i.e. exceeds background level). Ct levels are inversely proportional to the amount of target nucleic acid in the sample.

Certain embodiments of the present disclosure are further described in the following Examples. It should be understood that these Examples are given by way of illustration only. From the above discussion and these Examples, one skilled in the art can ascertain the essential characteristics, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### EXAMPLES

### Example 1: Preparation of Solution for Urine Sample Storage

Acetic acid-sodium acetate buffer (2 mol/L, pH=6.0), SDS solution (10% (MN)) and EDTA solution (0.5 mol/L, pH 8.4) were mixed at a ratio of 10:20:1 by volume to produce a solution for urine sample storage. For example, to prepare 310 mL solution, 100 ml of the acetic acid-sodium acetate buffer, 200 ml of the SDS solution, and 10 ml of the EDTA solution were mixed.

### Example 2: Preparation of Reagents for Urine Sample DNA Extraction

The following reagents were provided for extracting DNA from a urine sample:

Magnetic beads: Commercialized silicon hydroxyl magnetic beads with a particle size of 300 nm and a concentration of 50 mg/ml

Protease K: Commercially available 20 mg/ml proteinase K, diluted to 10 mg/ml with deionized water

Lysis solution: first preparing a solution comprising 5 M guanidinium isothiocyanate, 4% Triton X 100, 25 mM Tris-HCl (pH 6.5), 10 mM EDTA, and then adding to the solution 200% (V/V) dosage of isopropanol, and its final pH was adjusted to 6.5. The final lysis solution has 1.67 M guanidinium isothiocyanate, 1.33% Triton X 100, 8.33 mM Tris-HCl, 3.33 mM EDTA, and 66.7% (v/v of the lysis solution) isopropanol.

Washing buffer I: 50 mM isothiocyanate, 50 mM Tris-HCl (pH 5.0), 100 mM NaCl, and 60% ethanol and its final pH was adjusted to 5.0.

Washing buffer II: 10 mM Tris-HCl (pH 6.0) and 70% ethanol.

Elution buffer: 1×TE (pH 8.0).

### Example 3: Verification of Effectiveness of the Urine Sample Storage Reagent

Human urine samples were collected from multiple human subjects. Each urine sample was divided into 2 parts. The first part was added to a storage solution prepared in Example 1 in a ratio of 10:1 (urine sample: storage solution), and the second part was added with the same amount of sterile deionized water as a control. All samples were placed at 37 degrees Celsius for thermal acceleration experiments.

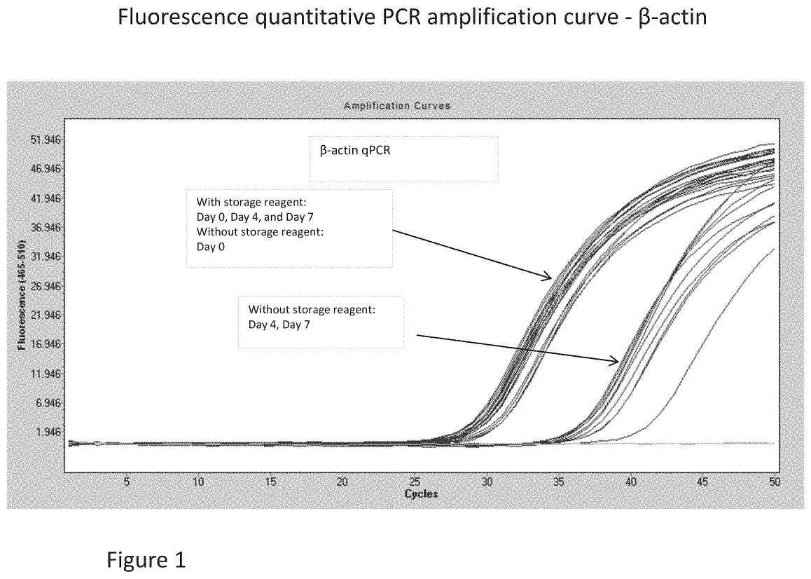

Samples were taken at 0, 4th, and 7th days, respectively. DNA in the collected samples was extracted using the urine DNA extraction reagent prepared in Example 2. (3-actin gene in the extracted DNA was amplified by quantitative PCR. The primers and probe sequences for detecting the (3-actin gene were: CGTGCTCAGGGCTTCTTGTC (upstream primer, SEQ ID NO: 1), CTCGTCGCCCACATAGGAATC, (downstream primer, SEQ ID NO: 2), and 5′-FAM-TGACCCATGCCCACCATCACGCCC-3′BHQ1 (probe, SEQ ID NO: 3). The results of the florescence quantitative PCR were used to determine DNA quality in the samples after the thermal acceleration experiments. The results are shown in Table 1 below and in FIG. 1.

TABLE 1
Validation of urine sample storage reagent

| | Day 0 | | | | | Day 4 | | | | | Day 7 | | | | |
|---|---|---|---|---|---|---|---|---|---|---|---|---|---|---|---|
| | β-actin Ct value | | | | | β-actin Ct value | | | | | β-actin Ct value | | | | |
| Control (w/storage reagent) | 29.67 | 29.83 | 29.8 | 29.68 | 29.58 | 36.9 | 36.58 | 38.04 | 37.29 | 36.79 | 37.41 | 41.04 | 36.34 | 37.04 | 38.07 |
| Test (w/storage reagent) | 29.18 | 29.5 | 29.2 | 29.08 | 29.29 | 30.63 | 30.26 | 30.89 | 30.58 | 30.7 | 29.34 | 28.94 | 29.3 | 28.97 | 28.85 |

The results indicated that, when urine samples mixed with the urine storage reagent were compared to urine samples collected at Day 0, there was no significant difference in terms of the (3-actin gene quantity and quality even after the urine samples were stored at 37° C. for 7 days, as verified by the qPCR Ct values. In contract, there was a significant difference between urine samples collected at Day 0 and urine samples stored 37° C. for just 4 days but without the storage reagent. The results showed that the urine storage reagent is effective in preserving DNA in the urine samples, even when the urine samples are stored at a high temperature.

### Example 4: Verification of stability of the urine sample storage reagent

This experiment was conducted to test DNA stability in urine samples after they are processed by the urine sample storage reagent produced in Example 1.

A group of high-risk HPV-positive urine samples collected from 12 human subjects was selected. Each urine sample was mixed with the urine storage reagent produced in Example 1 at a ratio of 10:1. Aliquots of each mixture were stored at 4° C. and room temperature.

DNA was extracted from these aliquots at 0, 1, 2, 3, and 4 weeks after the mixtures were made, using the DNA extraction reagent produced in Example 2. The DNA was used to detect DNA of HPV using a high-risk human papillomavirus detection kit (hybribio Bio), in order to determine stability of DNA in the urine samples.

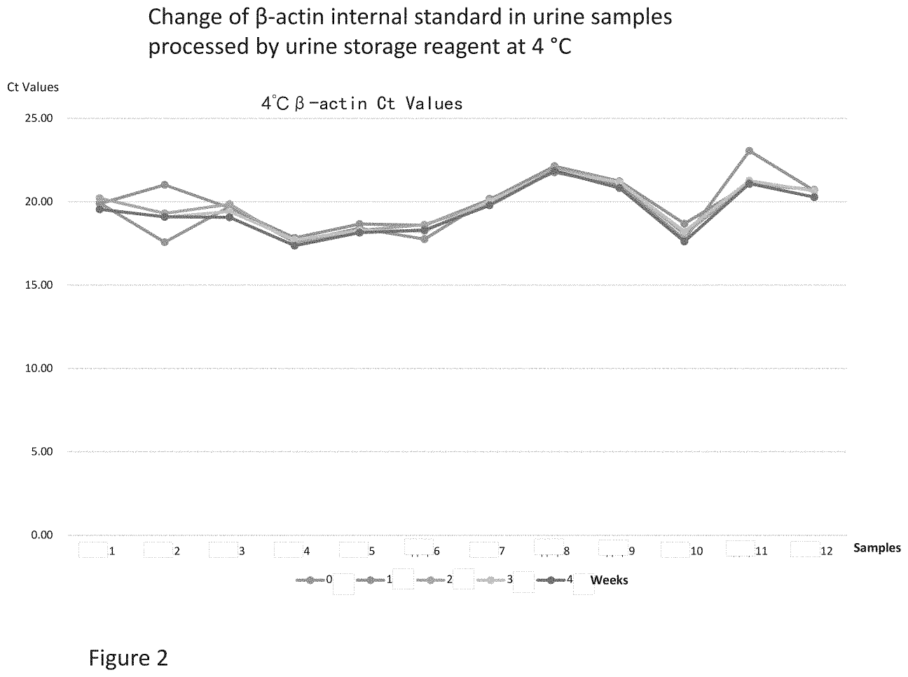

Table 2 and FIG. 2 demonstrate the stability of DNA of β-actin gene after 0, 1, 2, 3, and 4 weeks at 4° C., as indicated by Ct values of the β-actin gene in a fluorescence quantitative PCR.

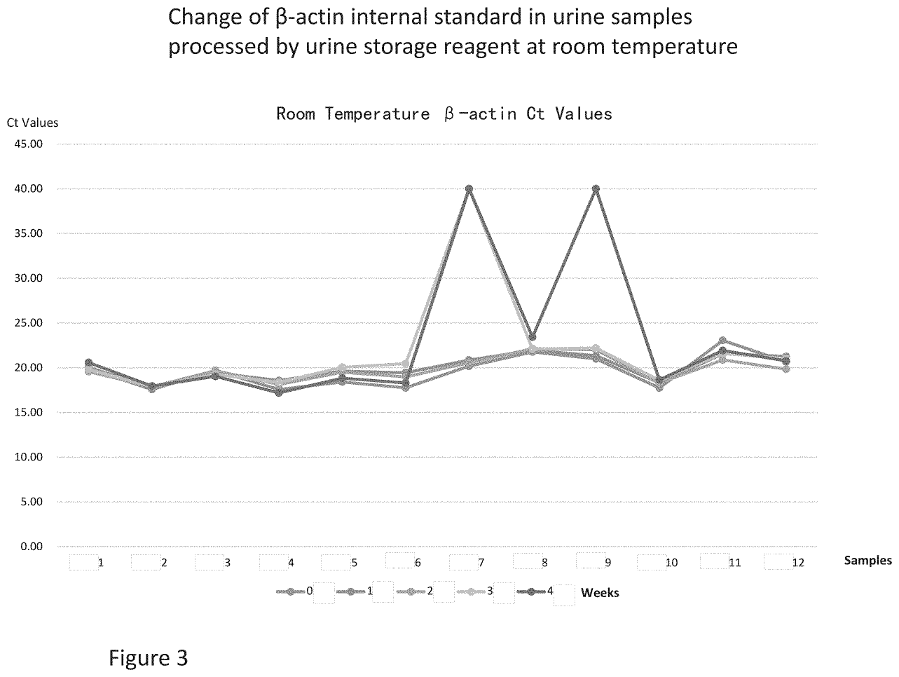

Table 3 and FIG. 3 demonstrate the stability of DNA of β -actin gene after 0, 1, 2, 3, and 4 weeks at room temperature, as indicated by Ct values of the β -actin gene in a fluorescence quantitative PCR.

TABLE 2
Stability of β-actin internal standard in urine samples w/storage reagent
at 4° C. (0 to 4 weeks, as indicated by qPCR Ct values)

| | Week 0 | Week 1 | Week 2 | Week 3 | Week 4 |
|---|---|---|---|---|---|
| | (ct value) | (ct value) | (ct value) | (ct value) | (ct value) |
| Sample 1 | 19.92 | 19.88 | 20.21 | 19.56 | 19.54 |
| Sample 2 | 17.57 | 21.01 | 19.30 | 19.08 | 19.09 |
| Sample 3 | 19.70 | 19.63 | 19.85 | 19.41 | 19.06 |
| Sample 4 | 17.57 | 17.83 | 17.55 | 17.71 | 17.36 |
| Sample 5 | 18.41 | 18.66 | 18.28 | 18.28 | 18.14 |
| Sample 6 | 17.75 | 18.58 | 18.62 | 18.21 | 18.30 |
| Sample 7 | 20.17 | 20.12 | 19.91 | 20.04 | 19.78 |
| Sample 8 | 21.76 | 22.13 | 22.01 | 21.85 | 21.85 |
| Sample 9 | 20.99 | 21.23 | 21.19 | 21.16 | 20.82 |
| Sample 10 | 17.74 | 18.68 | 18.05 | 18.22 | 17.61 |
| Sample 11 | 23.05 | 21.05 | 21.10 | 21.26 | 21.09 |
| Sample 12 | 20.66 | 20.71 | 20.72 | 20.64 | 20.27 |

TABLE 3
Stability of β-actin internal standard in urine
samples w/storage reagent at room temperature
(0 to 4 weeks, as indicated by qPCR Ct values)

| | Week 0 | Week 1 | Week 2 | Week 3 | Week 4 |
|---|---|---|---|---|---|
| | (ct value) | (ct value) | (ct value) | (ct value) | (ct value) |
| Sample 1 | 19.92 | 20.04 | 19.56 | 19.82 | 20.58 |
| Sample 2 | 17.57 | 17.82 | 17.87 | 17.88 | 17.95 |
| Sample 3 | 19.70 | 19.43 | 19.67 | 19.25 | 19.04 |
| Sample 4 | 17.57 | 18.57 | 18.13 | 18.36 | 17.19 |
| Sample 5 | 18.41 | 19.61 | 19.49 | 20.05 | 18.83 |
| Sample 6 | 17.75 | 19.44 | 18.98 | 20.46 | 18.30 |
| Sample 7 | 20.17 | 20.86 | 20.56 | 40.00 | 40.00 |
| Sample 8 | 21.76 | 21.95 | 22.13 | 22.07 | 23.43 |
| Sample 9 | 20.99 | 21.35 | 22.00 | 22.20 | 40.00 |
| Sample 10 | 17.74 | 18.31 | 18.29 | 18.60 | 18.64 |
| Sample 11 | 23.05 | 21.56 | 20.87 | 21.60 | 21.92 |
| Sample 12 | 20.66 | 21.26 | 19.84 | 21.01 | 20.76 |

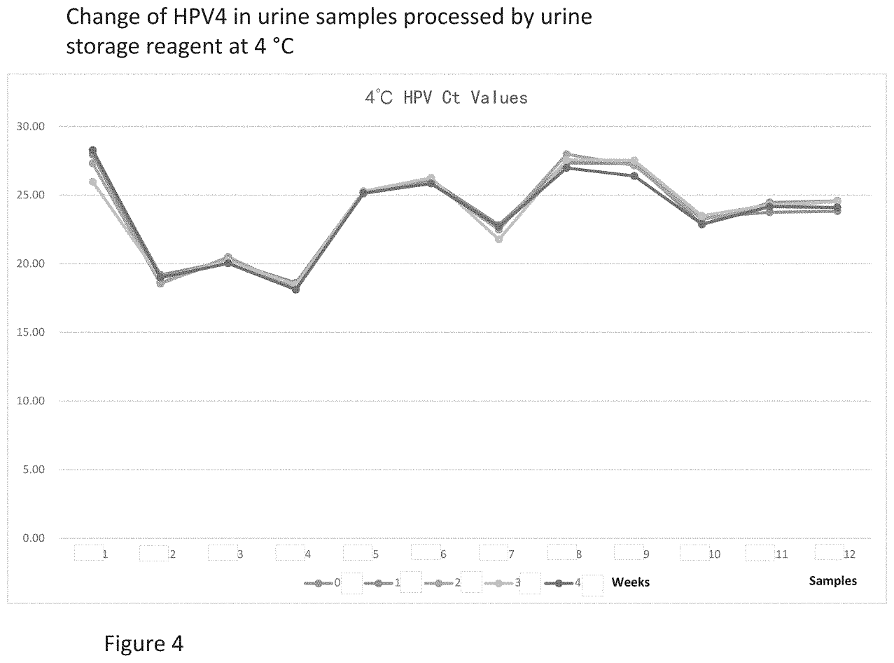

Table 4 and FIG. 4 demonstrate the stability of DNA of HPV marker gene after 0, 1, 2, 3, and 4 weeks at 4° C., as indicated by Ct values of the HPV marker gene in a fluorescence quantitative PCR.

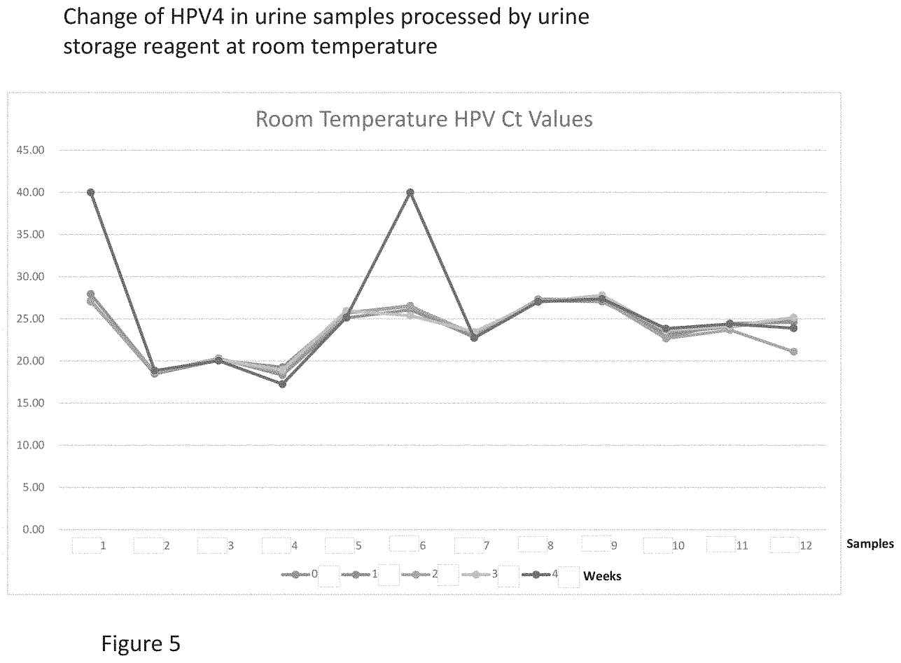

Table 5 and FIG. 5 demonstrate the stability of DNA of HPV marker gene after 0, 1, 2, 3, and 4 weeks at room temperature, as indicated by Ct values of the HPV marker gene in a fluorescence quantitative PCR.

TABLE 4
Stability of HPV marker gene in urine samples w/storage reagent
at 4° C. (0 to 4 weeks, as indicated by qPCR Ct values)

| | Week 0 | Week 1 | Week 2 | Week 3 | Week 4 |
|---|---|---|---|---|---|
| | (ct value) | (ct value) | (ct value) | (ct value) | (ct value) |
| Sample 1 | 27.95 | 28.14 | 27.32 | 25.97 | 28.28 |
| Sample 2 | 18.62 | 19.19 | 18.54 | 18.96 | 18.99 |
| Sample 3 | 20.30 | 20.18 | 20.49 | 20.20 | 20.03 |
| Sample 4 | 18.33 | 18.61 | 18.22 | 18.47 | 18.11 |
| Sample 5 | 25.11 | 25.27 | 25.11 | 25.29 | 25.16 |
| Sample 6 | 26.07 | 26.08 | 25.89 | 26.26 | 25.84 |
| Sample 7 | 22.83 | 22.59 | 22.48 | 21.77 | 22.69 |
| Sample 8 | 27.33 | 27.46 | 27.98 | 27.57 | 26.98 |
| Sample 9 | 27.30 | 27.30 | 27.15 | 27.52 | 26.39 |
| Sample 10 | 22.85 | 23.38 | 23.23 | 23.49 | 22.88 |
| Sample 11 | 24.46 | 23.75 | 24.14 | 24.31 | 24.16 |
| Sample 12 | 24.58 | 23.83 | 24.58 | 24.52 | 24.10 |

TABLE 5
Stability of HPV marker gene in urine samples w/storage reagent at
room temperature (0 to 4 weeks, as indicated by qPCR Ct values)

| | Week 0 | Week 1 | Week 2 | Week 3 | Week 4 |
|---|---|---|---|---|---|
| | (ct value) | (ct value) | (ct value) | (ct value) | (ct value) |
| Sample 1 | 27.95 | 27.15 | 27.03 | 40.00 | 40.00 |
| Sample 2 | 18.62 | 18.48 | 18.67 | 18.76 | 18.86 |
| Sample 3 | 20.30 | 20.08 | 20.11 | 20.16 | 20.03 |
| Sample 4 | 18.33 | 19.26 | 18.72 | 18.94 | 17.25 |
| Sample 5 | 25.11 | 25.71 | 25.65 | 25.96 | 25.19 |
| Sample 6 | 26.07 | 26.55 | 26.48 | 25.38 | 40.00 |
| Sample 7 | 22.83 | 22.76 | 23.19 | 23.45 | 22.73 |
| Sample 8 | 27.33 | 27.12 | 27.22 | 27.01 | 27.02 |
| Sample 9 | 27.30 | 27.05 | 27.23 | 27.80 | 27.37 |
| Sample 10 | 22.85 | 23.30 | 22.66 | 23.60 | 23.84 |
| Sample 11 | 24.46 | 24.06 | 23.68 | 24.13 | 24.39 |
| Sample 12 | 24.58 | 24.95 | 21.09 | 25.16 | 23.89 |

The above experimental results indicate that, after the urine samples were mixed with a urine storage reagent of the present disclosure, DNA molecules of the human (3-actin gene and the high-risk HPV DNA in the urine samples stored at 4° C. for 1 week, 2 weeks, 3 weeks, 4 weeks were comparable to DNA in urine samples collected at week 0, as there was no significant change in terms of DNA quality and quantity as measured by qPCR. For urine samples stored at room temperature after they were mixed with a urine storage reagent of the present disclosure, there was no significant change after one or two weeks compared to samples collected at week 0, but the samples began to become unstable after 3 or 4 weeks. The results suggest that a urine storage reagent of the present disclosure preserve DNA in a urine sample stable for at least 4 weeks when the processed samples are stored at 4° C., or at least 2 weeks at room temperature.

### Example 5: Verification of Effectiveness of DNA Extraction Reagents for Urine Samples

DNA in urine samples containing high-risk HPV were extracted by using several different methods/kits. Said methods/kits include Quick-DNA Urine Kit (ZYMO RESEARCH, D3061), magnetic bead urinary genomic DNA extraction kit (Enriching biotechnology, UDE-5005), FineMag large-volume magnetic bead—DNA extraction kit for plasma free DNA (Genefine Biotech, FM107), and the urine DNA extraction reagent of the present disclosure. After DNA extraction, the DNA was subjected to real-time quantitative PCR detection of HPV, using the high-risk human papillomavirus detection reagent of Hybribio. The instructions in each of the tested kits were followed.

To extract DNA from urine samples using DNA extraction reagents of the present disclosure, the following steps were taken:

1. Pretreatment of urine sample: 10 ml of urine sample was added into a 50 ml centrifuge tube. 20 μl of hydroxyl magnetic beads was added into the sample and mixed by vortexing. The tube was centrifuged for 5 min at 10000 rpm. Afterwards, supernatant was carefully discarded, and 500 μl of pellet was placed in a new 1.5 ml centrifuge tube. 2.5 μl of proteinase K was mixed with the pellet. The tube was heated in a metal bath at 56° C. for 30 min.

2. Extraction reagent dispensing: The lysis solution, washing buffer I, washing buffer II, and the elution buffer were added to a 96-well deep well extraction plate in a volume of 750 μl, 600 μl, 600 μl, and 50 respectively.

Table 6 demonstrated a possible sample loading plan. Among them, for each of the 8 rows A to H, two samples can be held for DNA extraction. For a 96-well plate, DNA from 16 samples can be extracted.

TABLE 6
Sample loading plan for DNA extraction on a 96-well plate.

| | 1 | 2 | 3 | 4 | 5 | 6 | 7 | 8 | 9 | 10 | 11 | 12 |
|---|---|---|---|---|---|---|---|---|---|---|---|---|
| A | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| B | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| C | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| D | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| E | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| F | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| G | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |
| H | Lysis | Lysis | Washing | Washing | | elution | Lysis | Lysis | Washing | Washing | | elution |
| | solution | solution | Buffer I | Buffer II | | buffer | solution | solution | Buffer I | Buffer II | | buffer |

750 μl of the lysis solution and 250 μl of the above pretreated urine sample were mixed in each well of columns 1, 2, 7, and 8. 600 μl of washing buffer I was added into each well of columns 3 and 9. 600 μl of washing buffer II was added into each well of columns 4 and 10. 50 μl of the elution buffer was added into each well of columns 6 and 12.

3. DNA Extraction using an automated DNA extraction equipment: The above described 96-well containing samples were placed into an automated DNA extraction equipment (Xi'An Tian Long, model NP968-S). Based on the manufacture manual, the following program was used:

TABLE 7
Program for automated DNA extraction equipment

| | Mixing | | Magnetic | | | Vortexing | | Hole | | Waiting |
|---|---|---|---|---|---|---|---|---|---|---|
| Step | time | | treatment time | Volume | | speed | Temp | position | Description | time |
| 1 | 10 | min | 60 s | 1000 | μl | Level 7 | | 1 | Lysis; binding | |
| 2 | 5 | min | 60 s | 1000 | μl | Level 7 | | 2 | Lysis; binding | |
| 3 | 3 | min | 60 s | 600 | μl | Level 7 | | 3 | Washing | |
| 4 | 2 | min | 60 s | 600 | μl | Level 7 | | 4 | Washing | |
| 5 | 5 | min | 60 s | 50 | μl | Level 7 | 65° C. | 6 | Elution | 5 min |
| 6 | 2 | min | | 50 | μl | Level 6 | | 4 | Remove magnetic | |
| | | | | | | | | | particles | |

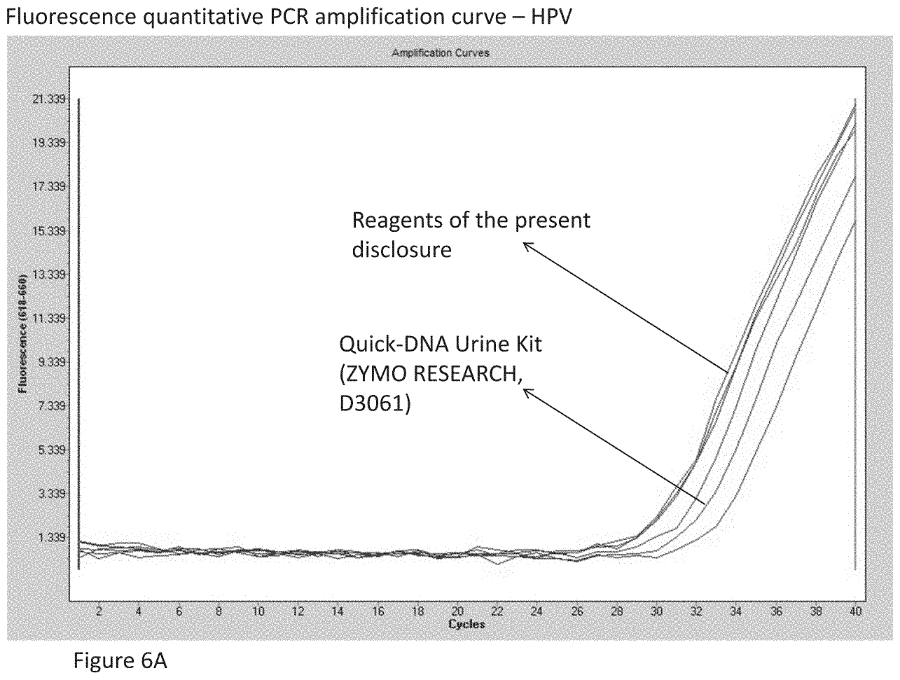

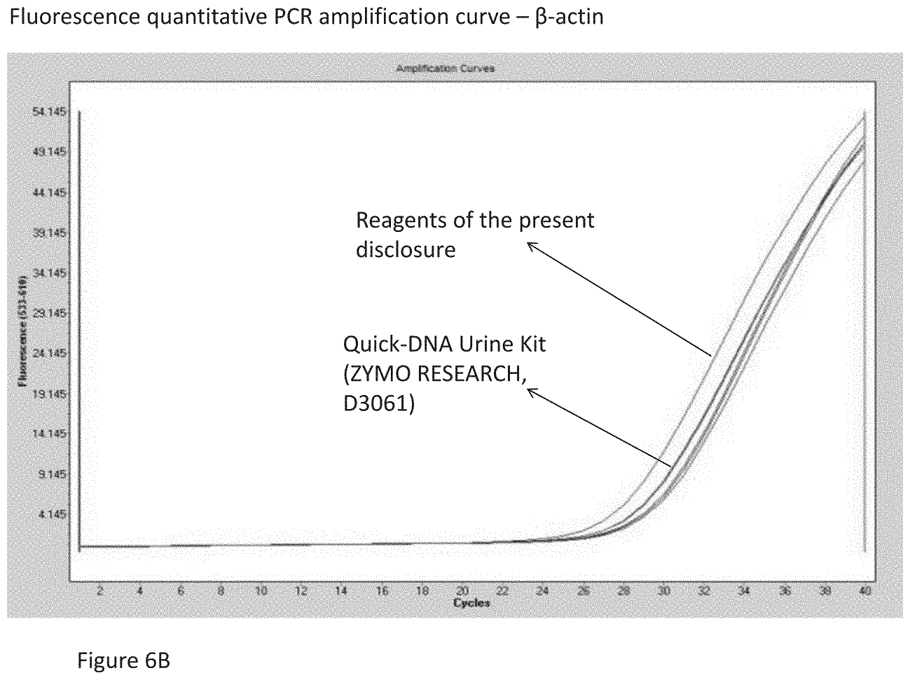

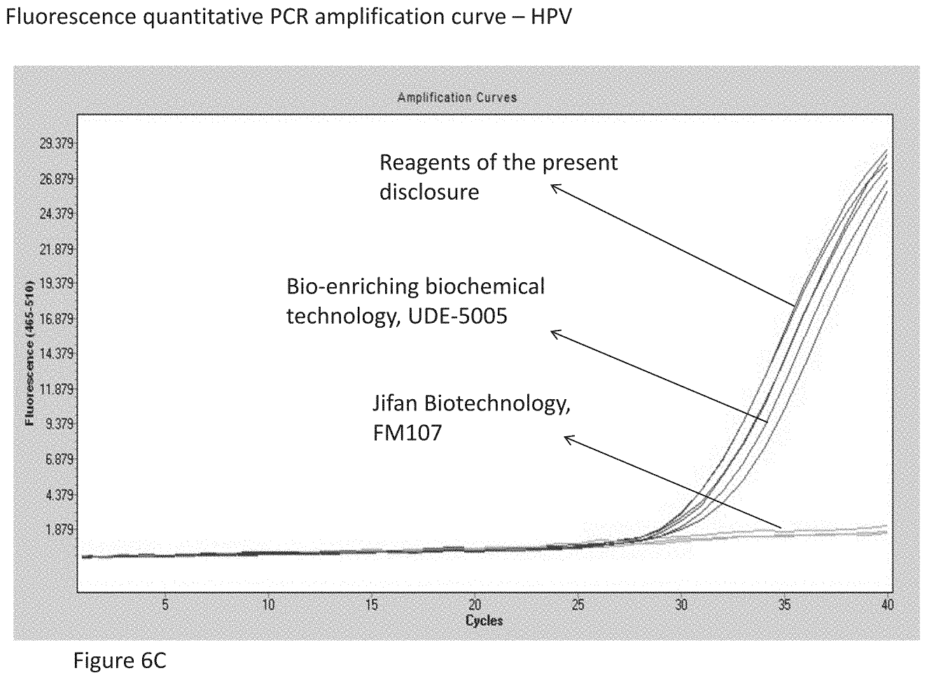

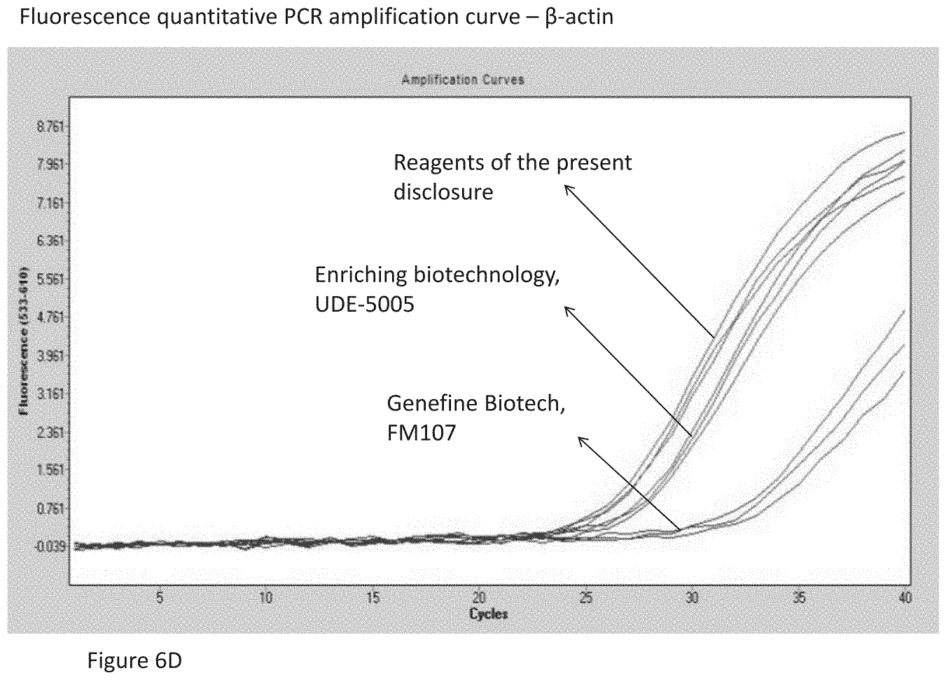

After DNA molecules were extracted from the urine samples using these different methods/kits, the extracted DNA molecules were used to detect HPV gene using fluorescence quantitative PCR, in order to determine DNA extraction efficiencies associated with each of these methods/kits. The same amount of urine sample was used for each DNA extraction method/kit, and the extracted DNA in each method was diluted to the same volume for PCR so that a meaningful comparison could be made. The results are shown in FIG. 6A to FIG. 6D.

The results indicate that reagents and methods of the present disclosure provide a more effective way to extract DNA from urine samples compared to the existing commercial products that were tested.

### Example 6: Optimizing the Formulation of Urine DNA Extraction Reagent

In order to improve the efficiency of extraction and purification of DNA extraction in the urine, formulations and/or dosage of the Lysis solution, Washing Buffer I, Washing Buffer II, dosage of magnetic beads and protease K were optimized.

Optimization of the Lysis solution: with the constant concentration of guanidine isothiocyanate at 5M and the dosage of isopropanol at 200%, the formulations of Lysis solution which contain 4% TritonX-100 with 5 different concentrations of EDTA (5 mM, 10 mM, 25 mM, 50 mM, 100 mM) were tested, and the formulations of Lysis solution which contain 10 mM EDTA with 5 different concentrations of Triton X-100 (1%, 2%, 4%, 6%, 8%) were tested. The “concentration” as used herein refers to the concentration of each component in the solution prior to adding isopropanol. In some embodiments, isopropanol is added after all other components are mixed together. DNA extraction was performed on the same urine sample using these Lysis solutions of different formulations (see Table 8). Extraction of DNA from urine sample was performed according to the method in Example 3 of the present invention, with 75% ethanol as washing buffer, 1*TE as elution buffer, 300 nm hydroxy magnetic beads, and 10 mg/ml protease K concentration. Quantitative PCR amplification was performed on the β -actin genes in the urine extracted from the Lysis solution of different formulations, and the extraction efficiency of the Lysis solution of different formulations was determined by the content of β -actin genes (which was inversely proportional to the measured Ct value). The primers and probe sequences for detecting the β -actin gene were: CGTGCTCAGGGCTTCTTGTC (upstream primer, SEQ ID NO: 1), CTCGTCGCCCACATAGGAATC, (downstream primer, SEQ ID NO: 2), and 5′-FAM-TGACCCATGCCCACCATCACGCCC-3′BHQ1 (probe, SEQ ID NO: 3). The results are shown in Table 9.

TABLE 8
Formulations of Lysis solution

| | | Triton | | | | isopropyl | |
|---|---|---|---|---|---|---|---|
| | GuSCN | X-100 | Tris-HCl | EDTA | | alcohol(V/V) | pH |
| Formulation 1 | 5M | 4% | 25 mM | 5 | mM | 200% | 6.5 |
| Formulation 2 | 5M | 4% | 25 mM | 10 | mM | 200% | 6.5 |
| Formulation 3 | 5M | 4% | 25 mM | 25 | mM | 200% | 6.5 |
| Formulation 4 | 5M | 4% | 25 mM | 50 | mM | 200% | 6.5 |
| Formulation 5 | 5M | 4% | 25 mM | 100 | mM | 200% | 6.5 |
| Formulation 6 | 5M | 1% | 25 mM | 10 | mM | 200% | 6.5 |
| Formulation 7 | 5M | 2% | 25 mM | 10 | mM | 200% | 6.5 |
| Formulation 8 | 5M | 4% | 25 mM | 10 | mM | 200% | 6.5 |
| Formulation 9 | 5M | 6% | 25 mM | 10 | mM | 200% | 6.5 |
| Formulation 10 | 5M | 8% | 25 mM | 10 | mM | 200% | 6.5 |

TABLE 9
β-actin gene testing results for different formulations
of Lysis solution used to extract DNA from urine samples

| Formulation | Test number | β-actin C_{T} | C_{T} mean |
|---|---|---|---|
| Formulation 1 | 1st | 32.09 | 31.74 |
| | 2nd | 31.86 | |
| | 3rd | 31.88 | |
| | 4th | 31.49 | |
| | 5th | 31.40 | |
| Formulation 2 | 1st | 32.57 | 31.21 |
| | 2nd | 25.56 | |
| | 3rd | 33.15 | |
| | 4th | 32.21 | |
| | 5th | 32.57 | |
| Formulation 3 | 1st | 31.79 | 31.83 |
| | 2nd | 32.06 | |
| | 3rd | 31.91 | |
| | 4th | 31.93 | |
| | 5th | 31.48 | |
| Formulation 4 | 1st | 31.60 | 31.94 |
| | 2nd | 32.23 | |
| | 3rd | 31.67 | |
| | 4th | 31.98 | |
| | 5th | 32.22 | |
| Formulation 5 | 1st | 32.29 | 31.81 |
| | 2nd | 32.44 | |
| | 3rd | 31.62 | |
| | 4th | No ct | |
| | 5th | 30.91 | |
| Formulation 6 | 1st | 31.96 | 31.84 |
| | 2nd | 31.65 | |
| | 3rd | 32.00 | |
| | 4th | 31.86 | |
| | 5th | 31.72 | |
| Formulation 7 | 1st | 32.96 | 32.13 |
| | 2nd | 31.75 | |
| | 3rd | 32.06 | |
| | 4th | 32.13 | |
| | 5th | 31.74 | |
| Formulation 8 | 1st | 32.18 | 32.22 |
| | 2nd | 32.13 | |
| | 3rd | 32.00 | |
| | 4th | 32.54 | |
| | 5th | 32.26 | |
| Formulation 9 | 1st | 31.98 | 32.04 |
| | 2nd | 31.96 | |
| | 3rd | 32.49 | |
| | 4th | 32.11 | |
| | 5th | 31.63 | |
| Formulation 10 | 1st | 31.88 | 31.80 |
| | 2nd | 31.98 | |
| | 3rd | 31.73 | |
| | 4th | 31.62 | |
| | 5th | 31.77 | |

As shown in Table 9, Formulation the β-actin gene content is the highest (the CT value is the lowest) in the DNA extracted from the formulation 2 lysis solution, so the Triton X-100 and EDTA concentration in the lysis solution are set as 4% and 10 mM, respectively.

A similar method was used to optimize the remaining components of the lysis solution. For guanidine thiocyanate the concentration gradient tested was 2 M, 3 M, 4 M, and 5M; For Tris-HCl the concentration gradient tested was 10 mM, 25 mM, 50 mM, and 100 mM; For the dosage of isopropanol (V/V) the gradient tested was 50%, 100%, 150%, 200% dosage; For the PH value setting a gradient of 5.5, 6.0, 6.5, 7.0 was tested. Finally, the optimal formulation for each component of the lysis solution in the invention is obtained as follows: 5M different guanidine thiocyanate, 4% TritonX-100, 25 mM Tris-HCl, 10 mM EDTA, pH=6.5. The “concentration” as used in this example refers to the concentration of each component in the solution prior to adding isopropanol.

Optimization of the washing buffer I: eight different formulations of washing buffer I were prepared according to Table 10 which, combined with other components of the urine DNA extraction reagent, were then applied to the same urine sample for sample extraction, and qPCR was used to evaluate β-actin genes content (following the method described in “Optimization of the Lysis solution”). The results are shown in Table 11.

TABLE 10
Eight different formulations of washing buffer I

| | | 50 mM | | | | ethyl |
|---|---|---|---|---|---|---|
| | GuSCN | Tris-HCl | NaCl | CTAB | PVP40 | alcohol |
| | (M) | pH | (M) | (%) | (%) | (%) |
| Formulation 1 | 0.5 | 6.0 | 0.10 | 0.01 | | 40 |
| Formulation 2 | 0.5 | 6.0 | 0.10 | / | 0.1 | 40 |
| Formulation 3 | 0.05 | 6.0 | 0.10 | / | 0.1 | 40 |
| Formulation 4 | 0.05 | 6.0 | 0.10 | / | 0.1 | 50 |
| Formulation 5 | 0.05 | 5.0 | 0.10 | / | 0.1 | 60 |
| Formulation 6 | / | 5.0 | 0.10 | / | | 40 |
| Formulation 7 | / | 5.0 | 0.10 | / | | 60 |
| Formulation 8 | 0.5 | 6.0 | 0.15 | / | 0.2 | 40 |

TABLE 11
β-actin gene testing results for different formulation
of Washing Buffer I used to extract DNA from urine samples

| Formulation | Testing number | β-actin C_{T} |
|---|---|---|
| Formulation 1 | 1^{st} | 21.66 |
| | 2^{nd} | 21.6 |
| | 3^{rd} | 21.56 |
| Formulation 2 | 1^{st} | 21.84 |
| | 2^{nd} | 21.95 |
| | 3^{rd} | 21.89 |
| Formulation 3 | 1^{st} | 22.64 |
| | 2^{nd} | 22.65 |
| | 3^{rd} | 22.44 |
| Formulation 4 | 1^{st} | 21.63 |
| | 2^{nd} | 21.53 |
| | 3^{rd} | 21.57 |
| Formulation 5 | 1^{st} | 21.52 |
| | 2^{nd} | 21.57 |
| | 3^{rd} | 21.56 |
| Formulation 6 | 1^{st} | 22.6 |
| | 2^{nd} | 22.63 |
| | 3^{rd} | 22.6 |
| Formulation 7 | 1^{st} | 21.59 |
| | 2^{nd} | 21.62 |
| | 3^{rd} | 21.6 |
| Formulation 8 | 1st | 21.72 |
| | 2nd | 21.73 |
| | 3rd | 21.79 |

According to the data analysis of table 11, formulation 5 of washing buffer I had the best extraction effect. Also, under this condition, the magnetic beads did not agglomerate in the extraction process, and the washing effect was better. Finally, the formulation of washing buffer I was determined as 0.05M GuSCN, 0.1% PVP40, 50 mM Tris-HCl, 60% ethanol, 100 mM NaCl, and pH=5.0.

Optimization of the washing buffer II: 75% ethanol (pH 6.0) containing 10 mM Tris-HCl (New Formulation) was prepared and compared with 75% ethanol (Original Formulation). Each buffer was combined with magnetic beads and other components of urine DNA extraction reagent, to extract DNA from 3 urine samples, respectively.

qPCR was used to evaluate the β-actin gene content (following the method described in “Optimization of the Lysis solution”) to check if the DNA loss during washing could be reduced. The results are shown in Table 12.

TABLE 12
Comparison of testing results of different washing buffer II

| Formulation | Testing number | β-actin C_{T} |
|---|---|---|
| Original Formulation | 1st | 23.50 |
| | 2nd | 24.25 |
| | 3rd | 24.52 |
| New Formulation | 1st | 23.63 |
| | 2^{nd} | 23.72 |
| | 3^{rd} | 23.63 |

According to the data analysis of Table 12, adjusting the pH value of 75% ethanol to pH 6.0 can reduce the DNA loss during washing, so the washing buffer II formulation is determined to be 75% ethanol, 10 mM Tris-HCl, pH=6.0.

Optimization of the magnetic bead dosage: the magnetic bead dosages were set at three different levels: 10 ul, 15 ul, and 20 ul. Each dosage of magnetic bead was combined with the remaining components of urine DNA extraction reagent for sample extraction from 2 urine samples accordingly for β-actin qPCR evaluation (following the method as described in “Optimization of the Lysis solution”). The results are shown in Table 13.

TABLE 13
Comparison of detection results of
different magnetic beads dosages

| magnetic beads dosage | Testing number | β-actin C_{T} |
|---|---|---|
| 15 ul | 1st | 22.54 |
| 15 ul | 2nd | 22.49 |
| 10 ul | 1st | 22.66 |
| 10 ul | 2nd | 22.64 |
| 20 ul | 1st | 21.98 |
| 20 ul | 2nd | 22.14 |

According to the data analysis of Table 13, increasing the amount of magnetic beads to 20 ul can improve the extraction efficiency, and the phenomenon of magnetic bead agglomeration can be eliminated in the extraction process. Therefore, the amount of magnetic beads was determined to be 20 ul.

Optimizing of the protease K dosage: protease K dosages were set at three levels: 0 ug, 2.5 ug and 25 ug. Each dosage of protease K was combined with the remaining components of urine DNA extraction reagent for sample extraction from 3 urine samples, which were then tested for (3-actin gene content with qPCR (following the method described in “Optimization of the Lysis solution”). The results are shown in Table 14 below.

TABLE 14
Comparison of test results of different protease K dosages

| | | |
|---|---|---|
| protease K dosage | Testing number | β-actin C_{T} |

| | | | |
|---|---|---|---|
| 0 | μg | 1^{st} | 23.48 |
| | | 2^{nd} | 23.24 |
| | | 3^{rd} | 23.61 |
| 2.5 | μg | 1^{st} | 21.33 |
| | | 2^{nd} | 21.53 |
| | | 3^{rd} | 21.54 |
| 25 | μg | 1^{st} | 21.49 |
| | | 2^{nd} | 20.9 |
| | | 3^{rd} | 21.16 |

According to the data analysis of Table 14, increasing the amount of protease K dosage to 25 ug can improve the extraction efficiency. Therefore, the amount of protease K dosage was finally determined to be 25 ug.

Optimization and determination of sample dosage: three clinical urine samples were selected, and for each urine sample the sample dosages (volumes) were tested at three levels: 400 ul, 1000 ul, and 8000 ul. The urine DNA extraction reagent and method described in the invention were used for DNA extraction and tested for β-actin gene with qPCR (following the method described in “Optimization of the Lysis solution”) to determine the optimal sample dosage. The results are shown in Table 15.

TABLE 15
Comparison of test results of different sample dosages

| | | |
|---|---|---|
| sample dosage | sample | β-actin C_{T} |

| | | | |
|---|---|---|---|
| 400 | μl | clinical urine sample1 | No ct |
| 1000 | μl | | No ct |
| 8000 | μl | | 37.23 |
| 400 | μl | clinical urine sample2 | 39.23 |
| 1000 | μl | | 36.97 |
| 8000 | μl | | 33.8 |
| 400 | μl | clinical urine sample3 | 33.28 |
| 1000 | μl | | 33 |
| 8000 | μl | | 29.5 |

According to the data analysis of Table 15, increasing sample dosage can significantly improve the detection result, with the 8000 μl sample size displaying the best result among these 3 sample dosages. In order to facilitate the operation, the sample dosage is finally set as 10 mL.

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

SEQUENCE LISTING

upstream primer, β-actin
SEQ ID NO: 1,
CGTGCTCAGGGCTTCTTGTC,
downstream primer, β-actin
SEQ ID NO: 2,
CTCGTCGCCCACATAGGAATC,
qPCR probe, β-actin
SEQ ID NO: 3,
5′-FAM-TGACCCATGCCCACCATCACGCCC-3′BHQ1

1. A composition for storing a urine sample obtained from a subject, wherein the composition comprises a pH buffer, a chelating agent, and a surfactant.

2. The composition of claim 1, wherein the pH buffer is configured to adjust a pH of the composition to within a preselected range.

3. The composition of claim 1, wherein the pH buffer comprises acetic acid and a salt of acetic acid.

4. The composition of claim 3, wherein the salt of acetic acid is sodium acetate.

5. The composition of claim 2, wherein the preselected range of pH is about 5.0 to 6.5.

6. The composition of claim 5, wherein the pH of the composition is about 6.0.

7. The composition of claim 4, wherein the sodium acetate has a concentration of about 0.5 to 1.0 mol/L.

8. The composition of claim 1, wherein the chelating agent is an aminopolycarboxylic acid.

9. The composition of claim 8, wherein the chelating agent is ethylenediaminetetraacetic acid (EDTA).

10. The composition of claim 8, wherein the EDTA has a concentration of about 10 to 25 mmol/L.

11. The composition of claim 1, wherein the surfactant is an anionic surfactant.

12. The composition of claim 11, wherein the anionic surfactant is a salt of dodecyl hydrogen sulfate.

13. The composition of claim 11, wherein the salt is a sodium salt, and the anionic surfactant is sodium docecyl sulfate (SDS).

14. The composition of claim 1, wherein the SDS has a concentration of about 5% to 10% (m/v).

15. The composition of claim 1, wherein the composition does not contain a preservative, a cell fixative, or a formaldehyde quencher.

16. A processed urine sample for storage, wherein the processed urine sample comprises a urine sample collected from a subject, a pH buffer, a chelating agent, and a surfactant.

17. The processed urine sample for storage of claim 16, wherein the pH buffer is configured to adjust a pH of the composition to within a preselected range.

18. The processed urine sample for storage of claim 16, wherein the pH buffer comprises acetic acid and a salt of acetic acid.

19. The processed urine sample for storage of claim 18, wherein the salt of acetic acid is sodium acetate.

20. The processed urine sample for storage of claim 17, wherein the preselected range of pH is about 5.0 to 6.5.

21. The processed urine sample for storage of claim 20, wherein the pH of the composition is about 6.0.

22. The processed urine sample for storage of claim 19, wherein the sodium acetate in the processed urine sample has a concentration of about 0.05 to 0.1 mol/L.

23. The processed urine sample for storage of claim 1, wherein the chelating agent is an aminopolycarboxylic acid.

24. The processed urine sample for storage of claim 23, wherein the chelating agent is ethylenediaminetetraacetic acid (EDTA).

25. The processed urine sample for storage of claim 24, wherein the EDTA has a concentration of about 1 to 2.5 mmol/L.

26. The processed urine sample for storage of claim 16, wherein the surfactant is an anionic surfactant.

27. The processed urine sample for storage of claim 26, wherein the anionic surfactant is a salt of dodecyl hydrogen sulfate.

28. The processed urine sample for storage of claim 26, wherein the salt is a sodium salt, and the anionic surfactant is sodium docecyl sulfate (SDS).

29. The processed urine sample for storage of claim 16, wherein the SDS has a concentration of about 0.5% to 1.5% (m/v).

30. The processed urine sample of claim 16, wherein the processed urine sample does not contain a preservative, a cell fixative, or a formaldehyde quencher.

31. A method for producing a processed urine sample for storage, comprising mixing a urine sample collected from a subject with a pH buffer, a chelating agent, and a surfactant, or with a composition of any one of claims 1 to 15.

32. The method of claim 31, wherein the pH buffer comprises acetic acid and a sodium acetate.

33. The method of claim 32, wherein the sodium acetate in the processed urine sample has a concentration of about 0.05 to 0.1 mol/L.

34. The method of claim 31, wherein the chelating agent is EDTA.

35. The method of claim 34, wherein the EDTA in the processed urine sample has a concentration of about 1 to 2.5 mmol/L.

36. The method of claim 31, wherein the surfactant is SDS.

37. The method of claim 36, wherein the SDS in the processed urine sample has a concentration of about 0.5% to 1.5% (m/v).

38. A method for storing a urine sample collected from a subject, comprising mixing the urine sample collected from the subject with a pH buffer, a chelating agent, and a surfactant to produce a urine sample ready for storage.

39. The method of claim 38, wherein the pH buffer, the chelating agent, and the surfactant are provided in a mixture before they are mixed with the urine sample collected from the subject.

40. The method of claim 39, wherein the pH buffer is configured to adjust a pH of the composition to within a preselected range.

41. The method of claim 38, wherein the pH buffer comprises acetic acid and a salt of acetic acid.

42. The method of claim 41, wherein the salt of acetic acid is sodium acetate.

43. The method of claim 40, wherein the preselected range of pH is about 5.0 to 6.5.

44. The method of claim 43, wherein the pH of the composition is about 6.0.

45. The method of claim 42, wherein the sodium acetate in the urine sample ready for storage has a concentration of about 0.05 to 0.1 mol/L.

46. The method of claim 38, wherein the chelating agent is an aminopolycarboxylic acid.

47. The method of claim 46, wherein the chelating agent is ethylenediaminetetraacetic acid (EDTA).

48. The method of claim 47, wherein the EDTA in the urine sample ready for storage has a concentration of about 1 to 2.5 mmol/L.

49. The method of claim 38, wherein the surfactant is an anionic surfactant.

50. The method of claim 49, wherein the anionic surfactant is a salt of dodecyl hydrogen sulfate.

51. The method of claim 50, wherein the salt is a sodium salt, and the anionic surfactant is sodium docecyl sulfate (SDS).

52. The method of claim 51, wherein the SDS in the urine sample ready for storage has a concentration of about 0.5% to 1.5% (m/v).

53. The method of claim 38, wherein the urine sample ready for storage does not contain a preservative, a cell fixative, or a formaldehyde quencher.

54. The method of claim 38, wherein the urine sample collected from the subject contains cells of the subject and at least one viral pathogen, and both the cells and the viral pathogen are lysed after the urine sample is ready for storage.

55. The method of claim 54, wherein the viral pathogen is a Human papillomavirus (HPV).

56. The method of claim 38, comprising storing the urine sample ready for storage at 4° C.

57. The method of claim 38, comprising storing the urine sample ready for storage at room temperature.

58. The method of claim 56, wherein DNA content in the urine sample is stable after a 15-day to 30-day storage time.

59. The method of claim 57, wherein DNA content in the urine sample is stable after a 1-week to 2-week storage time.

60. A method for detecting the presence or absence of one or more analytes in a urine sample collected from a subject, wherein the method comprises using a processed urine sample of any one of claims 16 to 30.

61. The method of claim 60, wherein the analyte is a virus.

62. The method of claim 61, wherein the virus is a HPV.

63. The method of claim 61, wherein the detection of the analyte comprises detecting DNA of the virus.

64. A kit for extracting DNA from a urine sample of a subject, wherein the kit comprises a lysis solution, magnetic nanoparticles, a protease, a first washing buffer, a second washing buffer, an elution buffer, or any combination thereof.

65. The kit of claim 64, wherein the lysis solution comprises guanidinium isothiocyanate, Triton X-100, Tris-HCl, EDTA, or any combination thereof.

66. The kit of claim 65, wherein the guanidinium isothiocyanate has a concentration of about 2 to 6 M, the Triton X-100 has a concentration of about 1 to 5%, the Tris-HCl has a concentration of about 20 to 50 mM, the lysis solution has a pH of about 6.5, the EDTA has a concentration of about 10 to 50 mM, or any combination thereof.

67. The kit of claim 66, wherein the lysis solution comprises guanidinium isothiocyanate, Triton X-100, Tris-HCl, and EDTA.

68. The kit of claim 66, wherein the lysis solution further comprises isopropanol.

69. The kit of claim 68, wherein a dosage of isopropanol is about 50% to 200% (v/v).

70. The kit of claim 69, wherein the guanidinium isothiocyanate has a concentration of about 1 to 2 M, the Triton X-100 has a concentration of about 1 to 2%, the Tris-HCl has a concentration of about 5 to 10 mM, the lysis solution has a pH of about 6-7, the EDTA has a concentration of about 3 to 5 mM, the isopropanol has a volume of about 50% to 80% of the lysis solution, or any combination thereof.

71. The kit of claim 64, wherein the magnetic nanoparticles have an inner core layer and an outer shell layer, wherein the inner core layer is composed of core-shell type magnetic nanoparticles, wherein the outer shell layer is composed of SiO₂.

72. The kit of claim 71, wherein the magnetic nanoparticles have a diameter of about 100 to 1000 nm, and a concentration of about 50 mg/ml.

73. The kit of claim 72, wherein the magnetic nanoparticles have a volume of about 10-20 μL.

74. The kit of claim 64, wherein the first washing buffer comprises guanidinium isothiocyanate, Tris-HCl, NaCl, and ethanol.

75. The kit of claim 74, wherein the guanidinium isothiocyanate has a concentration of about 50 mM.

76. The kit of claim 74, wherein the Tris-HCl has a concentration of about 20 to 50 mM,

77. The kit of claim 76, wherein the first washing buffer has a pH of about 5.0.

78. The kit of claim 74, wherein the NaCl has a concentration of about 50 to 200 mM.

79. The kit of claim 74, wherein the ethanol has concentration of about 40% to 60% (v/v).

80. The kit of claim 64, wherein the second washing buffer comprises Tris-HCl and ethanol.

81. The kit of claim 80, wherein the Tri-HCl in the second washing buffer has a concentration of about 10 to 50 mM, and the second washing buffer has a pH of about 6.0.

82. The kit of claim 80, wherein the ethanol has concentration of about 70% to 80% (v/v).

83. The kit of claim 64, wherein the elution buffer is a Tris-EDTA buffer having a pH of about 8.0.

84. The kit of claim 64, wherein the protease is protease K.

85. The kit of claim 84, wherein the protease K has a concentration of about 10 to 20 mg/ml.

86. The kit of claim 85, wherein the protease K has a dosage of about 2.5-25 μg.

87. A method for extracting DNA from a urine sample of a subject, comprises using a kit of any one of claims 64 to 86.

88. A method for extracting DNA from a urine sample of a subject, comprises:
(1) contacting the urine sample with magnetic nanoparticles and a protease to produce a pre-treated urine sample;
(2) lysing the pre-treated urine sample obtained in step (1) in a lysis solution to produce a lysed urine sample;
(3) washing the magnetic nanoparticles obtained in step (2) with a first washing buffer;
(4) washing the magnetic nanoparticles obtained in step (3) with a second washing buffer;
(5) collecting magnetic nanoparticles in the urine sample obtained in step (4); and
(6) washing off DNA from the collected magnetic nanoparticles obtained in step (5) with an elution buffer to obtain extracted DNA.

89. The method of claim 88, wherein the lysis solution comprises guanidinium isothiocyanate, Triton X-100, Tris-HCl, EDTA and isopropanol,
wherein the guanidinium isothiocyanate has a concentration of about 1 to 2 M;
wherein the Triton X 100 has a concentration of about 1 to 2%;
wherein the Tris-HCl has a concentration of about 5 to 10 mM and the lysis solution has a pH of about 6-7;
wherein the EDTA has a concentration of about 3 to 5 mM; and
wherein the isopropanol has a volume of about 50% to 80% (v/v) of the lysis solution.

90. The method of claim 88, wherein the magnetic nanoparticles have an inner core layer and an outer shell layer, wherein the inner core layer is composed of core-shell type magnetic nanoparticles, wherein the outer shell layer is composed of SiO₂, and the magnetic nanoparticles have a diameter of about 100 to 1000 nm, and a concentration of about 50 mg/ml.

91. The method of claim 88, wherein the first washing buffer comprises guanidinium isothiocyanate, Tris-HCl, NaCl, and ethanol,
wherein the guanidinium isothiocyanate has a concentration of about 50 to 100 mM;
wherein the Tris-HCl has a concentration of about 20 to 50 mM, wherein the first washing buffer has a pH of about 5.0;
wherein the NaCl has a concentration of about 50 to 200 mM;
wherein the ethanol has concentration of about 40% to 60% (v/v).

92. The method of claim 88, wherein the second washing buffer comprises Tris-HCl and ethanol.
wherein the Tri-HCl in the second washing buffer has a concentration of about 10 to 50 mM,
wherein the second washing buffer has a pH of about 6.0, and
wherein the ethanol has concentration of about 70% to 80% (v/v).

93. The method of claim 88, wherein the elution buffer is a Tris-EDTA buffer having a pH of about 8.0.

94. The method of claim 88, wherein the protease is protease K, wherein the protease K has a concentration of about 10 to 20 mg/ml.

95. The method of claim 88, wherein step (1) comprises
(a) contacting the urine sample with the magnetic nanoparticles to form a mixture;
(b) centrifuging the mixture or utilizing magnetic separation device to form a precipitate and a supernatant;
(c) contacting the precipitate with the protease to form a reaction system; and
(d) heating the reaction system under suitable conditions for a predetermined time.

96. The method of claim 88, wherein steps (3), (4), and/or (6) comprise using a magnetic frame or an automatic nucleic acid extraction instrument.

97. A method for detecting the presence or absence of an analyte in a urine sample collected from a subject, wherein the method comprises using DNA extracted from the urine sample using a kit of any one of claims 85 to 86.

98. The method of claim 97, wherein the analyte is a virus.

99. The method of claim 98, wherein the virus is a HPV.

100. The method of claim 98, wherein the detection of the analyte comprises detecting DNA of the virus.

101. A method for detecting the presence or absence of an analyte in a urine sample collected from a subject, wherein the method comprises:
(1) using a processed urine sample of any one of claims 16 to 30; and
(2) extracting DNA from the processed urine sample, comprising:
(a) contacting the urine sample with magnetic nanoparticles and a protease to produce a pre-treated urine sample;
(b) lysing the pre-treated urine sample obtained in step (a) in a lysis solution to produce a lysed urine sample;
(c) washing the magnetic nanoparticles obtained in step (b) with a first washing buffer;
(d) washing themagnetic nanoparticles obtained in step (c) with a second washing buffer;
(e) collecting magnetic nanoparticles in the urine sample obtained in step (d); and
(f) washing off DNA from the collected magnetic nanoparticles obtained in step (e) with an elution buffer to obtain extract DNA.

102. The method of claim 101, wherein the lysis solution comprises guanidinium isothiocyanate, Triton X-100, Tris-HCl, EDTA, and isopropanol,
wherein the guanidinium isothiocyanate has a concentration of about 1 to 2 M;
wherein the Triton X 100 has a concentration of about 1 to 2%;
wherein the Tris-HCl has a concentration of about 5 to 10 mM and the lysis solution has a pH of about 6-7;
wherein the EDTA has a concentration of about 3 to 5 mM; and
wherein the isopropanol has a volume of about 50% to 80% (v/v) of the lysis solution.
